SEQUENCE LISTING (3 sequences)

SEQ ID NO: 1 - DNA, 25 bp, Artificial Sequence
  Description of Artificial Sequence Synthetic oligonucleotide
gcgagtttttttttttttttctcgc

SEQ ID NO: 2 - DNA, 16 bp, Artificial Sequence
  Description of Artificial Sequence Synthetic oligonucleotide
gaaaaaaaaaaaaaaa

SEQ ID NO: 3 - DNA, 25 bp, Artificial Sequence
  Description of Artificial Sequence Synthetic oligonucleotide
gagcgtttttttttttttttctcgc

(19) United States
(12) Patent Application Publication
(10) Pub. No.: US 2015/0362500 A1
(43) Pub. Date: Dec. 17, 2015

(54) X-ray Excited Optical Materials and Methods for High Resolution Chemical Imaging

(71) Applicant: Clemson University, Clemson, SC (US)

(72) Inventors: Jeffrey Anker, Clemson, SC (US); Chen Hongyu, Clemson, SC (US); Frank Alexis, Greenville, SC (US)

(21) Appl. No.: 14/795,399

(22) Filed: Jul. 9, 2015

Related U.S. Application Data
(63) Continuation of application No. 13/612,061, filed on Sep. 12, 2012.

(51) Int. Cl. G01N 33/58; A61B 5/145; A61M 31/00; A61B 5/1455; A61K 49/04; A61B 6/00

(52) U.S. Cl. CPC G01N 33/582; A61K 49/0423; A61B 6/481; A61B 6/4216; A61M 31/005; A61B 5/1455; A61B 5/14539; G01N 2458/30

(57) ABSTRACT

X-ray/optical imaging materials are described and techniques as may be used for sensitive and high spatial resolution chemical and biophysical imaging in tissue. The technique uses high spatial resolution deeply penetrating X-rays to excite scintillators which convert the energy to a different frequency, e.g., visible light frequencies. The emitted spectrum is then modulated by a chemical indicating element such as an indicator dye held in optical communication with the scintillators in order to detect specific concentrations in the local area. The materials can include a magnetic element in conjunction with the scintillator and chemical indicating element. The materials can incorporate a biologically active agent for delivery.

### CROSS REFERENCE TO RELATED APPLICATION

This application is a continuing application of U.S. patent application Ser. No. 13/612,061, having a filing date of Sep. 12, 2012, which claims filing benefit of U.S. Provisional Patent Application Ser. No. 61/534,437 having a filing date of Sep. 14, 2011, which is incorporated herein in its entirety by reference.

### STATEMENT REGARDING FEDERALLY SPONSORED RESEARCH OR DEVELOPMENT

This invention was made with government support under Grant No. DMR-0755005 awarded by the National Science Foundation, under Award No. NNG05GI68G awarded by the National Air and Space Administration through the South Carolina Space Grants Palmetto Academy Program, and under Grant Nos. 5P20RR021949 and 8P20GM103444 awarded by National Institute of Health to The Center of Biomaterials for Tissue Regeneration. The government has certain rights in the invention.

### SEQUENCE LISTING

The instant application contains a Sequence Listing which has been submitted electronically in ASCII format and is hereby incorporated by reference in its entirety. Said ASCII copy, created on Aug. 26, 2015, is named CXU-687-CON(2011-011)_SL.txt and is 1,106 bytes in size.

### BACKGROUND

Although optical microscopy is a powerful technique for chemical analysis in biological samples and a wide variety of chemically sensitive fluorescent and absorptive dyes are available for non-invasive, sensitive, specific, rapid, and high-resolution chemical imaging, its use is severely hindered in tissues deeper than 1 millimeter (mm) as almost all light is scattered (no ballistic photons). In addition, high concentrations of indicator dyes are required to overcome background autofluorescence and even for background-free techniques (e.g. chemiluminescence) scattering reduces the ability to pinpoint a source to a few millimeters in as little as 1 centimeter (cm) of tissue. For instance, in theoretical models of homogeneous slabs of highly scattering tissue and in experimental homogenous slab phantoms, two point light sources may be resolved only when they are separated by approximately 0.2d or more (d=depth of object), even using lifetime gating to select for early arriving minimally scattered photons. In mouse bioluminescence experiments a point chemiluminescent source implanted approximately 6 mm into a mouse could be located only to within 1.5 mm of the true position (determined with micro-CT) using a sophisticated model of the mouse based on a mouse atlas, and only to within 3.5 mm using a homogenous tissue scattering model. Spatially resolving multiple luminescent sources in the tissue to forma an image is even more challenging than localizing this single source.

Several other methodologies permit high resolution imaging, but they lack chemical sensitivity. For instance, Magnetic Resonance Imaging (MRI) contrast provides high resolution imaging (as good as 10-100 micrometers (μm)), but does not generally provide molecular information. X-rays provide a unique ability to visualize differences in tissue density due to their long scattering depths (about 2.0 cm in tissue using 20 keV X-rays and 7.0 cm for 100 keV, according to NIST attenuation tables). Two-dimensional images can be obtained by using scintillators to measure the intensity of transmitted X-rays, and a 3D CT image can be obtained by rotating the sample to acquire transmission images at each angle and applying the Radon transform (or similar algorithm) to reconstruct the image. Conventional X-ray radiography is limited, however, in that its primary purpose is to detect changes in density rather than specific chemical concentrations. Positron emission tomography (PET) and single photon positron emission computer tomography (SPECT) are used to measure the distribution of radioactive analytes injected in vivo. This chemical imaging is limited, however, in that the analyte must be radiolabelled, the resolution is typically in millimeters, and half-lives are relatively short. Scintillation proximity assays measure concentration of radiolabelled molecules bound or in close proximity to scintillator particles and may be performed in vitro or in vivo with injected scintillators. While these assays are essentially background free, the resolution is limited by optical scattering.

Imaging the biochemistry in tissues is essential for understanding diseases and developing therapeutics. For example, chemical imaging of oxygen and pH is important for understanding tumor resistance to chemotherapy as tumor hypoxia and acidosis modulate the effectiveness of chemotherapy, photodynamic therapy, and X-ray therapy.

Improved imaging of biochemistry in tissues would also be of great benefit in diagnosis of infection as occurs in conjunction with device implantation. Implanted devices such as joint replacements, pacemakers, valve prosthesis, and synthetic vascular grafts are increasingly used improve lives. Unfortunately implants are highly susceptible to infection: 1-3% of the 600,000 joint prostheses implanted per year in the U.S. and 5-10% of the two million fixators have been estimated to become infected. In addition, approximately half of the two million infections acquired at hospitals in the U.S. are associated with implanted medical devices. Battlefield injuries to extremities can account for 75% of wartime trauma with high energy fractures of long bones being a predominant insult. External fixation systems and dynamic compression plate systems are used to treat such fractures prior to wound closure. These fracture fixation systems, directly applied to bone surfaces using screws proximal and distal to the fracture, are a low-cost, surgically simple solution to fracture stabilization. Introducing these pins, plates and screws, however, increases the potential for latent bacterial infection near these implant surfaces, especially in injuries with debris in the wounds, resulting in chronic infection rates estimated at 40% for battlefield injuries with internal fixation.

Bacteria easily colonize implanted device surfaces and are hard to eradicate once established. A review of diagnosis and treatment of prosthetic joint associated infections states that, “[t]he cornerstone of successful treatment is early diagnosis. Since treatment is less invasive . . . in patients with a short history of infection, delay in diagnosis should be avoided.” Unfortunately, infection is difficult to diagnose when bacteria are localized on the device surface, and the presentation of infections associated with implants can be a challenge, especially in patients with multiple potential infection sources. There is a risk of morbidity and even mortality from surgical intervention or prolonged hospitalization. This makes an incorrect diagnosis most undesirable. Early diagnosis of implant infection has proven difficult: CT imaging can detect bone resorption and sinus tracts but is unhelpful until late in the course of infection; markers of systemic inflammation are not specific; and bacteria are often localized to the implant surface and not always found in joint fluid.

Attempts have been made to form “smart implants,” that use electronic sensors for detecting chemical/biophysical changes on implanted medical devices upon bacterial infection. However, these devices require complex electronics for power, detection, data processing, and telemetry that reduces long term reliability. Also, miniaturization and attachment present problems for detection over large regions of irregular implant surfaces.

An in situ non-invasive technique for high resolution, high chemical sensitivity imaging as may be utilized in detecting local biochemistry so as to better diagnose implant bacterial infection as well as hypoxia, acidosis, and protease activity, among other conditions, would be of great benefit in diagnosis and treatment monitoring.

### SUMMARY

According to one embodiment, disclosed is a sensing system as may be utilized for high resolution imaging of an analyte, for instance in deep tissue visualization. The sensing system includes a scintillator that emits optical photons upon excitation by an X-ray. The sensing system also includes a chemical indicating element that is in optical communication with the scintillator. The chemical indicating element exhibits an optically detectable response to the scintillator emission. The optically detectable response of the chemical indicating element provides information with regard to the presence or amount of the analyte. For instance, the chemical indicating element modifies the optical spectrum of the scintillator in a manner that depends upon the local concentration of the analyte.

Also disclosed is a method for determining the presence or amount of an analyte in a turbid environment. The method can include directing an X-ray beam at the scintillator so as to excite the scintillator, leading to emission of a photon from the scintillator. The scintillator is in optical communication with the chemical indicating element that exhibits an optically detectable response to the scintillator emission. More specifically, the chemical indicating element can modify the optical spectrum of the scintillator in a manner that depends upon the local concentration of the analyte. The method also includes detecting the optically detectable response of the chemical indicating element, which provides information with regard to the presence or amount of the analyte in the environment.

### BRIEF DESCRIPTION OF THE FIGURES

A full and enabling disclosure of the subject matter, including the best mode thereof, directed to one of ordinary skill in the art, is set forth more particularly in the remainder of the specification, which makes reference to the appended figures in which:

FIG. 1 illustrates the fluorescence emission intensity of an oxygen chemical indicating element as may be incorporated in a sensing system as described herein.

FIG. 2 schematically illustrates an excitation/detection system as may be utilized in conjunction with the sensing system described herein.

FIG. 3 schematically illustrates a polynucleotide-based FRET sensing system as described herein (SEQ ID NOS 1-3, respectively, in order of appearance).

FIG. 4 is a schematic of a scanning X-ray technique as may be utilized in conjunction with a sensing system.

FIG. 5 is the spectra of a scintillator-containing film either coated or uncoated with a silver layer.

FIG. 6 schematically illustrates another scanning X-ray technique.

FIG. 7 includes the luminescence responses of a system as measured with a photomultiplier tube at A and B. The plot at C illustrates the log of the intensity vs. time after a chopper blocked the X-ray beam.

FIG. 8A illustrates extinction spectra of silver island film on cover glass (cover glass was used as the reference). All spectra are normalized to Aₘₐₓ=1. The real Aₘₐₓ for 1, 3, 5, 10, 20, 30, 40, 50, 60 nm are 0.02, 0.17, 0.31, 0.59, 0.84, 1.25, 1.60, 1.95, and 2.23, respectively.

FIG. 8B illustrates extinction spectra of silver island film on scintillator particles (scintillator film coated cover glass was used as the reference). All spectra are normalized to Aₘₐₓ=1. The real Aₘₐₓ for 1, 3, 5, 10, 20, 30, 40, 50, 60 nm are 0.02, 0.09, 0.14, 0.51, 0.55, 0.69, 0.83, 0.87, and 0.90, respectively. The inset shows a photograph of the silver coated scintillator film.

FIG. 9A illustrates the luminescent spectra of Gd₂O₂S:Eu scintillator coated with different thickness of silver film.

FIG. 9B illustrates that the relative luminescence loss of the system of FIG. 9A ((I_{uncoated}−I_{coated})/I_{uncoated}×100%) varies with different thickness of silver.

FIG. 10A illustrates the luminescent intensity of a system at 617 nm in air and oil.

FIG. 10B illustrates the relative luminescent intensity loss of the system of FIG. 10A ((I_{uncoated}−I_{coated})/I_{uncoated}×100%) in air and oil.

FIG. 11 illustrates the luminescent intensity of a system at 617 nm as a function of time during silver dissolution in 1 mM H₂O2 at 23° C.

FIG. 12 illustrates the luminescent spectra of scintillator films, one with 5 nm of silver film and one with 5 nm of gold film.

FIG. 13 illustrates at A and D images of gold and silver coated scintillator films before (A) and after (D) H₂O₂ etching, respectively. At B and E are illustrated the intensity of red light scanned at different positions with (B) and without (E) 10 mm of tissue before H₂O₂ etching. At C and F are illustrated the intensity of red light scanned at different positions with (C) and without (F) 10 mm of tissue after H₂O₂ etching. The resolution through 10 mm tissue was 1.7 mm.

FIG. 14 is a schematic illustration of a system as described herein.

FIG. 15A is a spectrum of red and green phosphors of the system of FIG. 14 and FIG. 15B shows the ratio of red and green light as a function of X-ray focal spot position with/without 1 cm of tissue. The resolution through tissue was 0.30 mm.

FIG. 16 is a photograph of a polyethylene knee implant with a sensing system as described herein embedded in the implant.

FIGS. 17A and 17B are X-ray diffraction patterns of Gd₂O₂S:Tb (green) scintillator (FIG. 17A) and Gd₂O₂S:Eu (Red) scintillator (FIG. 17B).

FIG. 18A illustrates a magnetically modulated fluorescence signal through 6 mm of chicken breast using green excitation and 565 nm emission.

FIG. 18B illustrates the effects of scattering (extinction on modulated intensity (left axis) as well as on background intensity (right axis) for magnetically modulated sensing systems as described herein.

FIG. 19 includes at A a flow diagram for a formation method for magnetic, hollow, core/shell particles including a scintillator. FIG. 19 includes at B a scanning electron microscopy (SEM) image of the particles formed according to the method of FIG. 24 and in the inset at B is a size distribution chart of the formed particles. FIG. 19 includes at C a transmission electron microscopy (TEM) image of the particles formed according to the method of FIG. 24.

FIG. 20 includes TEM images of core/shell particles incubated in 0.5 M oxalic acid at 60° C. for 8.5 h (A), 9.5 h (B), 10 h (C).

FIG. 21 includes at a. X-ray diffraction (XRD) patterns of (α-Fe₂O₃@SiO₂@Gd(OH)CO₃:Eu) particles, at b. γ-Fe₂O₃@SiO₂@Gd₂O₃:Eu particles (iron oxide core was incubated in oxalic acid for 9.5 h), and at c. iron oxide core particles incubated in oxalic acid for 9.5 h with a thin (˜10 nm) Gd₂O₃:Eu shell.

FIG. 22 includes TEM images of solid particles at A and hollow particles at B formed as described herein.

FIG. 23 includes the magnetic hysteresis loops of magnetic probes including (A) nanorice with maghemite as the core, (B) nanoeyes (iron oxide core was incubated in oxalic acid for 9.5 h), (C) nanorice with hematite as the core, and (D) hollow nanorice.

FIG. 24 includes at A and B a schematic presentation of magnetic modulation of scattering of light by nanoeyes (iron oxide core was incubated in oxalic acid for 9.5 h). FIG. 24 at C illustrates the intensity time series for magnetic nanoeyes under a darkfield microscope.

FIG. 25 illustrates the radioluminescence spectra of nanoeyes (iron oxide core incubated in oxalic acid for 9.5 h) and nanorice excited by X-ray at A, and fluorescence spectra of nanoeyes (iron oxide core incubated in oxalic acid for 9.5 h) and nanorice excited by 480 nm light at B.

FIG. 26 illustrates T₂ and T₂*-weighted images of solid nanorice (A and A*), nanoeyes (iron oxide core was incubated in oxalic acid for 9.5 h) (B and B*), and hollow nanorice (C and C*) at echo time of 4 ms and 1.5 ms, respectively as shown.

FIG. 27 illustrates the relaxation rate curves as a function of concentration for solid nanorice, nanoeyes, and hollow nanorice. Error bars represent the standard deviation.

FIG. 28 illustrates the results of cytotoxitity testing for hollow nanorice (Gd2O3:Eu).

FIG. 29 includes at A a schematic illustration of a synthesis of DOX@Gd₂O₂S:Tb@PSS/PAH delivery capsules and pH-responsive release of doxorubicin from the capsules. FIG. 29 includes at B a low magnification TEM image and at C a high magnification TEM image of DOX@Gd₂O₂S:Tb@PSS/PAH delivery capsules formed as described herein.

FIG. 30 graphically illustrates the results of cytotoxicity tests of Gd₂O₂S:Tb and Gd₂O₂S:Eu nanocapsules.

FIG. 31 illustrates the X-ray luminescence of Gd₂O₂S:Tb nanocapsules (A) and Gd₂O₂S:Eu nanocapsules (B).

FIG. 32 presents fluorescence spectra of Gd2O2S:Tb and Gd2O2S:Eu nanocapsules excited by 460 to 495 nm light

FIG. 33 Illustrates an image of nanocapsules having a solid core and including coated multilayers of PSS/PAH (A), and the absorbance of cumulative doxorubicin release from hollow nanocapsules and nanocapsules having a solid core at pH 2 (B).

FIG. 34 illustrates the cumulative release of doxorubicin from DOX@Gd2O2S:Tb@PSS/PAH at pH 5.0 and 7.4 (A), and the peak ratio of real time radioluminescence detection at 544 and 620 nm as a function of time at pH 5.0 and pH 7.4 (B).

FIG. 35 Illustrates the absorption of doxorubicin at pH 5.0 and 7.4 and the X-ray radioluminescence of Gd₂O₂S:Tb@PSS/PAH nanocapsules (A), and the real-time radioluminescent spectra of DOX@Gd₂O₂S:Tb@PSS/PAH at pH 5.0 at 0.5 hours, 12 hours, and 43 hours (B).

FIG. 36 illustrates T2 and T2*-weighted images of radioluminescent nanocapsules. Group A: T2-weighted images of Gd₂O₂S:Eu nanocapsules with concentration of 0.8 mg/ml, 0.4 mg/ml, 0.1 mg/ml, and 0.05 mg/ml. Group B: T2-weighted images of Gd₂O₂S:Tb with concentration of 0.8 mg/ml, 0.4 mg/ml, 0.1 mg/ml, and 0.05 mg/ml. Group A*: T2*-weighted images of Gd₂O₂S:Eu nanocapsules with concentration of 0.8 mg/ml, 0.4 mg/ml, 0.1 mg/ml, and 0.05 mg/ml. Group B*: T2*-weighted images of Gd₂O₂S:Tb nanocapsules with concentration of 0.8 mg/ml, 0.4 mg/ml, 0.1 mg/ml, and 0.05 mg/ml.

FIG. 37 presents a schematic illustration of an X-ray protease beacon that switches on and luminesces when and the protease cleaves the peptide sequence and a gold nanoparticle separates.

FIG. 38 illustrates the effects of gold nanoparticle quenching of radioluminescence. VIS/NIR spectrum and TEM image (inset) of Popovtzer's 30 nm gold nanoparticles. Radioluminescence spectrum and TEM image (inset) of anker's 50 nm radioluminescent Gd₂O₂S:Eu nanoparticles. Adsorption of Popovtzer's positively charged amino-PEG functionalized 30 nm gold nanoparticles to Anker's negatively charged radioluminescent Gd₂O₂S:Eu causes the radioluminescence intensity to decrease by a factor of 12 compared to control using Popovtzer's negatively charged citrate stabilized gold nanoparticles.

FIG. 39 presents the luminescence of different nanophosphors under different excitation source (A) Gd₂O₂S:Tb,Yb,Ho, (B) Gd₂O₂S:Tb@Gd₂O₂S:Yb, Ho, (C) Gd₂O₂S:Eu@Gd₂O₂S:Yb, Er.

FIG. 40 is a photograph of a microscope slide with drops at a series of different pHs.

FIG. 41 presents the extinction spectra of the pH sensing film as a function of pH.

FIG. 42 is an image of Staphylococcus epidermidis growing on pH sensing film, displaying local acidic regions.

FIG. 43 is a schematic presentation of a method for fluoride doping of nanoparticles.

FIG. 44 is a photograph of luminescence of X-ray and up-conversion nanophosphors before and after NaF doping under room light, X-ray excitation (for Gd₂O₂S:Tb and Gd₂O₂S:Eu) or 980 nm laser (for Gd₂O₂S: Yb,Er).

FIG. 45 presents the luminescence spectra for Gd₂O₂S:Tb (A), Gd₂O₂S:Eu (B) and Gd₂O₂S: Yb,Er (C).

### DETAILED DESCRIPTION OF REPRESENTATIVE EMBODIMENTS

Reference now will be made in detail to various embodiments of the disclosed subject matter, one or more examples of which are set forth below. Each example is provided by way of explanation, not limitation. In fact, it will be apparent to those skilled in the art that modifications and variations may be made in the present disclosure without departing from the scope or spirit of the subject matter. For instance, features illustrated or described as part of one embodiment may be used on another embodiment to yield a still further embodiment. Thus, it is intended that the present disclosure covers such modifications and variations as come within the scope of the appended claims and their equivalents.

In general, disclosed herein are hybrid X-ray/optical imaging materials and techniques as may be used for sensitive and high spatial resolution chemical and biophysical imaging in tissue. High resolution chemical imaging can facilitate biochemical studies of tissue, for instance to determine acidosis and chemotherapy resistance in tumors as well as in early detection of bacterial infection at implantation sites. The technique uses high spatial resolution deeply penetrating X-rays to excite scintillators which convert the energy to a different frequency, e.g., visible light frequencies. The emitted spectrum is then modulated by a chemical indicating element such as an indicator dye held in optical communication with the scintillators in order to detect specific concentrations in the local area.

Beneficially, a narrow focused X-ray beam can be used to excite the scintillators and thereby to define the source of illumination of the chemical indicating element with a resolution limited by the incident X-ray beam-width. The sensing system can be designed to have predetermined chemically modulated spectral and luminescence lifetimes. The luminescence spectrum can be utilized to indicate local chemical concentration, while the X-ray beam can specifically define the location of the targeted analyte. Detection can be carried out by use of an excitation/detection system. For instance, imaging can be accomplished by raster scanning the X-ray source relative to the sample. The disclosed systems and methods can combine the advantages of X-ray imaging with the versatile sensitivity of optical chemical indicators.

In one embodiment, the system can also include a magnetic element in conjunction with the scintillator and the chemical indicating element. Adding magnetic functionality to the system can provide several benefits. For instance, the addition of a magnetic element can enable components of the system to be guided, oriented, and heated using external magnetic fields. In addition, location and spectrum analysis can be imaged with X-ray luminescent tomography as well as complementary magnetic resonance imaging.

Several characteristics make this technology particularly beneficial. First, the hybrid X-ray excitation/optical detection method provides an in situ, background-free technique that combines the high resolution of X-ray imaging with the chemical sensitivity of optical chemical indicating elements such as dyes. Second, separation of the chemical indicating element from the X-ray scintillator light source can provide a highly robust and versatile modular design. Third, the use of phosphorescence lifetime imaging to avoid absorption artifacts can enable reliable readings even in complex and changing environments. Fourth, the technique can be complimentary to X-ray transmission imaging and optical microscopy, fluorescence imaging, and upconversion imaging which can be performed on the same imaging setup. Fifth, existing components can be assembled to make a field-portable device. Additional benefits will be evident to one of ordinary skill in the art.

Scintillators used in a sensing system can include X-ray phosphors as are generally known. A wide range of X-ray phosphors are available including, without limitation, NaI, CsI, CaWO₄, lanthanide halide scintillators doped with a rare earth dopant such as Ce, Tb, or Eu, Gd₂O₃, Eu:CdTe quantum dots, anthracene nanoparticles, and Tb labeled actin. In one embodiment, europium and cesium doped LaF₃ and LuF₃ nanoparticles can be utilized as these materials have a high quantum efficiency and reasonable stability. In another embodiment, Gd₂O₂S:Eu nanoparticles can be used that are also highly luminescent and can be fabricated with a wide range of sizes and shapes including core-shell particles with multiple functionalities to the cores and shells (e.g. an upconversion core, a spacer layer, and a radioluminescent shell).

In general, the scintillator can be in the form of a micro- or nano-sized particle. As utilized herein, a microparticle can general have an average diameter of less than about 900 micrometers (μm), less than about 500 μm, or less than about 100 μm. A nanoparticle generally is a particles having an average diameter of less than about 500 nanometers (nm), less than about 100 nm, less than about 50 nm, or less than about 20 nm that can exhibit high quantum efficiency, stability, and a relatively long lifetime to allow efficient energy transfer. In one embodiment, the particle can have a diameter of from about 5 nm to about 10 μm. Particles can generally be of any shape. For instance, particles can be generally circular, ovoid, amorphous, or spindle shaped. The shape of a particle can generally depend upon materials of formation and/or formation conditions.

The scintillator can exhibit an emission lifetime on the order of microseconds (μs) (e.g., from about 1 to about 5 μs for the rare earth donors). By way of example, europium has an emission lifetime of about 1 μs. In one embodiment, a relatively fast decaying scintillator can be incorporated in the system so as to distinguish the lifetime of the chemical indicating element from that of the scintillator (e.g., 60 μS for PtOEPK, and 1 millisecond (ms) for PdTSPP dyes). This is not a requirement, however, and in another embodiment a long lifetime scintillator can be used and the quenching lifetime of the chemical indicating element can be determined. For instance, the quenching lifetime due to interaction of the scintillator emission with a chemical indicating element that is an absorber acceptor molecule of the scintillator emission or is a plasmonic nanoparticle can be determined.

In one embodiment, the scintillator can be a component of a core or a shell of a core/shell particle. For example, a particle core can be formed of a magnetic material, such as an iron oxide core (e.g., a γ-Fe₂O₃ core) and a scintillator shell can be formed on the core. In this embodiment, the sensing system can also include a magnetic element as discussed above in addition to the other components of the system. In another embodiment, the core can be formed of the chemical indicating element and a scintillator shell can be formed on the core.

The scintillator (e.g., scintillator-containing micro- or nano-sized particles) can be combined with a chemical indicating element to form a sensing system. For instance, the chemical indicating element can be provided in a single particle in conjunction with the scintillator, e.g., in a shell formed on the particle. Alternatively, the chemical indicating element can be a separate material provided in conjunction with the scintillator, but not necessarily as a component of a single structure such as a core/shell particle.

During use of the sensing system, excitation of the scintillator by X-ray energy can cause an energy transfer first to the scintillator (e.g., a Ru, Ce, or Eu center) followed by energy transfer to, or absorption by, the chemical indicating element. The chemical indicating element can be any material that can exhibit an optically detectable response to the scintillator emission. In addition, the optically detectable response of the chemical indicating element can provide information with regard to the presence or amount of analyte in the local area of the system. For example, the chemical indicating element can be a fluorescent dye that emits a distinct spectrum in response to the scintillator emission when in the presence of a specific analyte. In another embodiment, the chemical indicating element can be an absorber of the scintillator emission, and the optically detectable quenching effect of the chemical indicating element can indicate information with regard to the presence or amount of the targeted analyte. In yet another embodiment, the chemical indicating element can be a biologically active analyte, for instance a drug that is delivered to the environment in conjunction with the scintillator, and the optically detectable response of the chemical indicating element can indicate successful delivery of the drug to the desired location. A chemical indicating element (e.g., a chemical indicating element that is embedded in an implantable device) can be an inorganic material and can have a relatively long operational lifetime, for instance as compared to a system based upon degradable or leachable organic dyes.

According to one embodiment, the chemical indicating element can be a pH indicator. In general, a pH indicator can be any material for which the emission spectrum under the influence of the scintillator emission can vary depending upon the pH of the local environment. pH indicators as may be utilized as a chemical indicating element in a system can include, without limitation, coumarin-based pH sensitive dyes, bromocresol green, phthalein type dyes, fluorescein type dyes, rhodamine type dyes, and so forth In one embodiment pH sensing can be carried out by use of the ratiometric indicator dye ETH5350 with fluorescence peaks at 600 nm and 660 nm or alternatively the more hydrophilic SNARF-SE dye with ratiometric peaks at 580 nm and 640 nm. Of course, other pH chemical indicating elements can alternatively be utilized in a system.

The system can be an oxygen indicator and can incorporate a chemical indicating element that can be utilized to indicate the presence or amount of oxygen in a local environment. By way of example, an oxygen sensor can be based upon colorimetric absorbance by hemoglobin or fluorescence excitation and oxygen quenching of ruthenium.

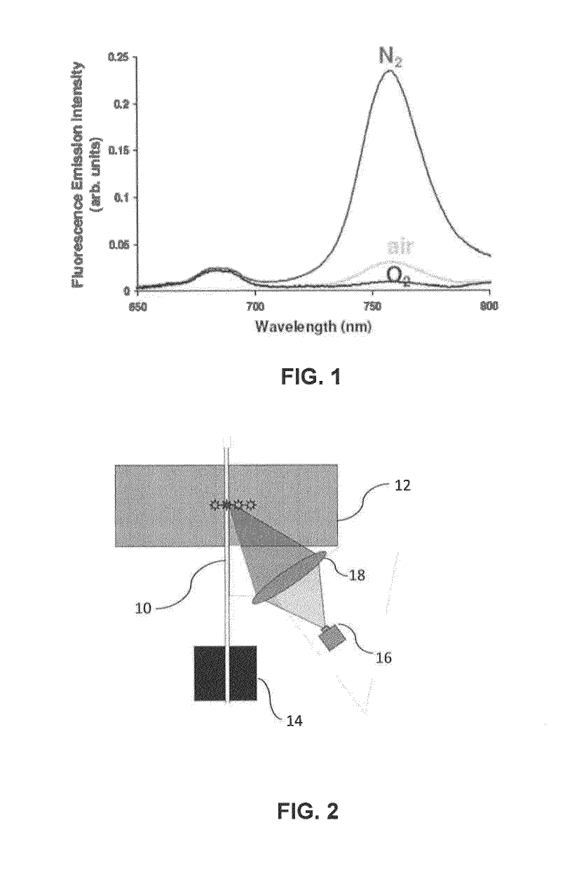

FIG. 1 illustrates the response of a silica-based nanoparticle including an oxygen sensitive platinum(II) octaethylporphine ketone (PtOEPK) dye as may be utilized in forming a system as described herein. In this embodiment, the system can function as an oxygen sensor based upon the fluorescence quenching of the chemical indicating element (i.e., the oxygen sensitive dye (760 nm emission peak)) that includes an unknown oxygen-insensitive impurity that exists persistently and serves here as a reference (685 nm emission peak). In this system, the ratio between the two peaks can provide a measure of the oxygen concentration that is independent of the concentration of the particles as well as the incident light intensity, which can be provided by X-ray excitation of the scintillator that is in optical communication with the chemical indicating element.

In one embodiment a plasmonic particle may be utilized as a chemical indicating element in a system. Plasmonic particles are attractive as absorptive chemical indicating elements with a spectrum that can be modulated by dissolution, or cleavage and disaggregation of polymer-bound nanoparticle dimmers and aggregation. For instance, gold or silver may be utilized as a chemical indicating element. Gold and silver are especially interesting materials for spectrochemical sensors because they support localized surface plasmon resonance (LSPR) modes, which provide intense size- and shape-dependent optical absorption and scattering spectra. Gold and silver are currently used in a wide variety of chemical sensors based upon refractive index changes as well as aggregation and disaggregation. Silver, particularly in the form of nanoparticles, has also been utilized as an antimicrobial agent and has been proposed as a method to prevent and reduce implant associated infection. The disclosed system can provide a route for the on-going determination of the in situ dissolution rate of silver, for instance on an implant surface. This system can be valuable for evaluating antimicrobial efficacy and silver toxicity and can provide an early diagnosis route for a microbial infection. For example the loss of the optically detectable signal of a silver chemical indicating element, for instance due to dissolution of the silver due to the presence of a bacterial infection, could be utilized as an early indication of the infection.

Gold and silver are also ideally suited to lithography techniques that can be utilized both for validating image resolution of a system as well as for providing spatially separated sensing regions on a device or in a system, for instance for determination of internal reference standards.

The dissolution of a silver chemical indicating element is an example of a chemical indicating element that is not permanently engaged in the system in conjunction with the scintillator. In this type of a system, the loss of the optically detectable signal from the chemical indicating element can be utilized to determine that the chemical indicating element is no longer located in optical communication with the scintillator, and this loss of signal can provide information regarding the presence or amount of analyte in the local environment. For example, in the case of a silver chemical indicating element, the loss of optically detectable signal from the chemical indicating element can signal bacterial infection in the area.

The chemical indicating element can be a material that can be delivered from the system, for instance a biologically active material such as a drug. There are pluralities of biologically active agents that can provide an optically detectable response to the scintillator emission as may be incorporated in a system. By way of example, doxorubicin, which is an anthracycline antibiotic commonly used as a cancer chemotherapy, is light sensitive and exhibits an optically detectable signal to an emission in the visible light spectrum. In one embodiment, a delivery vehicle (e.g., a hollow micro- or nano-sized particle) can include a scintillator and an optically detectable drug such as doxorubicin in optical communication with one another. Upon delivery of the doxorubicin at the desired location, the optically detectable signal from the delivery vehicle can change, signaling successful release of the drug from the vehicle. Other drugs that may be used include chemotherapy drugs such as Mitoxantrone and Mitomycin C, photodynamic therapy drugs such as Photofrin, Platinum(II) octaethylporphyrin, Platinum(II) octaethylporphyrin ketone, Zinc phthalocyanine, and methyl blue. In addition, release of drugs that do not have strong absorption spectra can be tracked by measuring concurrent release of tracer dyes with absorption spectra that overlap with the X-ray excited optical luminescence spectrum, or molecules that quench the luminescence. The spectral change upon drug release may be determined from changes in luminescence lifetime or the shape of the luminescence spectrum. A second set of scintillator particles with similar surface chemistry, size, and shape, but different rare earth dopants or host matricies may be used as a spectral reference with closely spaced spectral peaks to reduce spectral distortion from light propagation through the tissue. For example, Gd₂O₃:Sm, Gd₂O₃:Eu and Gd₂O₂S:Eu, have distinct spectral peaks at ˜610 nm that differ by less than 5 nm.

In another embodiment, a biologically active agent that is to be delivered from the system can be incorporated into the system in conjunction with a separate chemical indicating element. In this embodiment, the presence or amount of the biologically active agent may be indirectly determined through direct determination of the presence or amount of the optically detectable chemical indicating element. For instance, the chemical indicating element (e.g., a fluorescent indicator dye) can be loaded into a delivery vehicle in conjunction with a biologically active agent. During use, the chemical indicating element can be released from the delivery vehicle in conjunction with the biologically active agent. Thus, loss of signal of the chemical indicating element can signal successful delivery of the biologically active agent.

The surface of the nanoparticles can be modified to improve the circulation time, increase colloidal stability, reduce biofouling, target the particles to specific cells and organs, and/or control the release of drugs from the particles. This modification can include molecular recognition elements such as antibodies, aptamers, and antigens, as well as materials to reduce non-specific binding such as polyethylene glycol (PEG). The coating materials include but are not limited to synthetic polymers, petptides, proteins, nucleic acids, glycoproteins and lipids.

The invention is not limited to a specific method of loading the drug into. Many loading techniques are known to those skilled in the art, including precipitation, mircofluidic printing, emulsion methods, and so on. The fabrication process is not limited to a specific method, and many methods are known to those skilled in the art, including photolithographic techniques, imprint lithography techniques, template techniques, hydrothermal techniques, gas phase synthesis, and so on.

The specific design of the system is not particularly limited, with the only requirement being that the scintillator and the chemical indicating element can be in optical communication with one another through at least a portion of the utilization of the system. Beneficially, as the chemical indicating element and the scintillator are separate materials, the two components can be separately optimized for use in any particular application.

Two factors contribute to the determination of the optimal distance between a scintillator and a chemical indicating element. The first factor is the distance that allows for the fluorescence resonance energy transfer (FRET) between the scintillator and the chemical indicating element. In general, FRET is effective for donor/acceptor distances of less than about 10 nanometers, for instance between about 1 and about 10 nm. The second factor is the inner filter absorption effect, which can alter the spectrum and intensity of the optically detectable response of the system. Both factors depend on the overlap between the donor's emission spectrum and the chemical indicating element's excitation spectrum. Heavy atom quenching and energy transfer to molecular oxygen are other methods of affecting the luminescence lifetime which are known to those skilled in the art.

According to one embodiment, the scintillator can be in the form of nano-sized particles that are encapsulated in a material that can include the chemical indicating element. Encapsulation of the scintillator can be utilized to increase the uniformity and rapidity of the response of the system through control of the scintillator response (i.e., lifetime, intensity, and spectra). By way of example, nanoparticles can be encapsulated in a layer that includes silica combined with an indicator dye forming a core/shell nanoparticle including the scintillator in the core and the chemical indicating element in the shell.

A core/shell construction is not a requirement of a system, however, and any construction that provides the scintillator in optical communication with the chemical indicating element may alternatively be utilized. For instance, in one embodiment, both the scintillator and the chemical indicating element can be provided in the form of separate nanoparticles, and the two types of nanoparticles can be held in optical communication with one another, for instance in a polymeric film.

Films incorporating the scintillator and the chemical indicating element can be single layer or multilayer films. For instance, scintillator nanoparticles can be mixed with nanoparticles of the chemical indicating element, and the two types of nanoparticles can be extruded together in a melt-processed film. Alternatively, a multilayer film can be formed with adjacent layers containing the scintillator and the chemical indicating element, respectively. In one embodiment, silica films can be formed that include embedded pH dyes. The films can then be fabricated above a thin layer (e.g., about 15 micrometers) that includes X-ray scintillators. The multilayer film including the scintillators and chemical indicating element can then located as desired, for instance, the multilayer film can be placed within a polyethylene prosthetic implant.

A single or multilayer film can be designed so as to use identical surface chemistry to native implant surfaces. For example, the multilayer film can include a polymer matrix formed of the same polymer as is used to form the surface of an implant. In one embodiment, a chemical indicating element, for instance an oxygen sensing fluorescent dye, can be embedded directly beneath polyethylene that can be combined with a layer including the scintillators for use in a polyethylene-based implant. In another embodiment, chemical indicating elements may be embedded in conjunction with nano-sized scintillators in nanoporous titania films that can then be applied to titanium implants. Beneficially, and unlike CT and MRI, a metal substrate will not create artifacts when imaging the disclosed system.

In one embodiment, relatively thick layers of scintillator-containing material may be added to the surface of an implant or into holes of a porous implant allowing a large signal from a locally two-dimensional sample. This can simplify the mathematical problem of 3D optical tomography reconstruction, reduce the required exposure, increase the acquisition rate, and improve reproducibility. It can also support use of a simple and versatile detection methodology in which the optical chemical indicating element (e.g., a fluorescent or colorimetric dye) is placed in a separate layer adjacent the scintillator layer providing an inner filter-effect based sensor. Separating the light source (the scintillator) from the chemical indicating element can simplify the design and optimization of complex and multiplexed sensors. Possibility of toxicity can also be reduced by embedding the scintillators into the implant so that they interact only if the implant erodes, and then are released only slowly.

In one embodiment, the chemical indicating element can be held in a matrix through which the targeted analytes can diffuse. For example, oxygen diffuses through hydrophobic polymers relatively rapidly (e.g. 1.4×10⁻⁷ to 1.6×10⁻⁷ cm²/s through polypropylene membranes). In this embodiment, the chemical indicating element can be maintained within the implant over a long lifetime. This is an advantage for developing robust sensors. For instance, the chemical indicating elements and scintillators can be held in in implants including thin layers of a hydrophobic polymer, and the presence or amount of the targeted chemical (e.g., oxygen) can be determined over the course of the implant life. Diffusion of the targeted analyte within the implant can be expected to spread the oxygen signal by approximately the depth that it propagates, but this can be easily accounted for during utilization.

The sensing systems can be located in a plurality of areas throughout an implant. An important advantage of this embodiment is that imaging at multiple locations can increase redundancy. In addition, chemical indicating elements and scintillators can be embedded at various depths throughout an implant to protect against degradation and increase durability.

The system including the scintillator and the chemical indicating element can be utilized in conjunction with delivery of a biological agent. For instance, the system can include the scintillator and a chemical indicating element as a component of a delivery vehicle. In one embodiment, a delivery vehicle can be a hollow particle, for instance a hollow particle formed to include a scintillator as a component of the particle material, and the drug to be delivered from the particle can be located within the hollow particle. For example, the core of a core/shell particle can be partially or completely removed, as by an etching process, an example of which is provided in the Examples, set forth below, so as to form a hollow particle. The chemical indicating element can be located in the particle material in conjunction with the scintillator, in a shell layer adjacent to the particle material including the scintillator, or in the hollow center of the particle.

Of course, a delivery vehicle is not limited to a particle or a hollow particle and other delivery vehicles as are known in the art may be utilized in conjunction with the system. For example, an implantable device such as a stent, degradable scaffolding, or other type of implantable delivery device can incorporate a biologically active agent to be delivered from the implantable device. The biologically active agent can diffuse from the implantable device, can be released from the implantable device as the device degrades, or can be released according to any other methodology as is generally known. In one embodiment, an implantable device can include a surface coating that can degrade or otherwise alter in the environment in which the device will be located so as to allow release of the biologically active agent. For instance, the implantable device (e.g., a hollow nano-sized particle) can include a pH-sensitive coating. At the predetermined environmental conditions (for instance at acidic conditions), the coating can dissipate or otherwise alter such that the biologically active agent is released from the vehicle.

As previously discussed, in those embodiments in which the biologically active agent is optically active under the scintillator emission, the biologically active agent can also serve as the chemical indicating element. Alternatively, the implantable device can incorporate a separate chemical indicating element that can be utilized to indirectly determine information with regard to the presence or amount of the biologically active agent in the local area. For instance as the chemical indicating element is released from the implantable device, it can be inferred that the biologically active agent is being released in conjunction with the chemical indicating element. Alternatively, the chemical indicating element can be utilized to determine information with regard to the presence or amount of another analyte at the site, for instance the chemical indicating element can be pH sensitive for determination of the local pH.

A system can include multiple scintillators and/or multiple chemical indicating elements. For instance, many dyes and phosphors are available for multiple spectrally and spatially separated sensors on a single device. The inclusion of multiple scintillators and/or multiple chemical indicating elements can provide a route for reference sites on an implantable device that can be utilized for internal calibration and/or for determination of the presence or amount of a second analyte in the local area.

The system may also include a magnetic element, for instance as a component of a micro or nano-sized particle that includes the scintillator and/or the chemical indicating element. Multifunctional magnetic and fluorescent materials have attracted broad interest because of their utility in biomedical applications such as bioimaging, drug delivery carriers, magnetic resonance imaging (MRI), bio-separation, fluorescent labeling, magnetic hyperthermia, and immunoassays. These magnetic particles can be magnetically guided, oriented, heated, and imaged using external magnetic fields. Meanwhile the particle fluorescence can provide a sensitive label for imaging in cells and thin tissue sections. The particles' optical and magnetic properties can be varied depending upon the core size, which can be controlled by formation methods, for instance by varying the etching time. The etching process can also form a hollow space within or around the core which could be used for encapsulation of a material to be delivered from the system. Inclusion of a scintillator with such materials, for instance in a shell of a core/shell particle or as a separate particle in optical communication with the magnetic/optical particles, can provide a route to improved and novel utilizations. For instance, a magnetic element can provide a route for separation of materials in magnetic field gradients

In addition to magnetophoretic separation in magnetic field gradients, magnetic element containing particles can align with external magnetic fields due to their magnetic shape anisotropy. This alignment can be useful in developing sensitive and rapid immunoassays, viscosity sensors, and improved intracellular sensors with orientation and shape-dependent properties. For example, orientation of non-spherical particles can affect phagocytosis rates for adherent macrophages. In addition, the ability for particles to rotate can be useful for determining if particles are bound, while the rotation rate in solution can be useful for measuring viscosity and viscous drag. The magnetic radioluminescent particles described herein can also serve as T₂ and T₂* contrast agents in magnetic resonance imaging. The particles are also T₁ and T₁* contrast agents, with improved contrast for thin and porous nanostructures.

In detecting the optical response of the chemical indicating element to the scintillator emission, the effects of spectral distortion due to imaging through tissue can be resolved with spectral deconvolution or through the use of closely spaced or narrow spectral peaks. Spectral deconvolution is possible either by principle components analysis of luminescence, or by use of a reference spectrum from a scintillator with a different rare earth doping or the same type of scintillator but spaced at a distance, for instance about 100 μm or more away from the scintillator that is in optical communication with the chemical indicating element.

In an embodiment in which the system is utilized for visualization in tissue, the optically detectable signal of the chemical indicating element will generally pass through the tissue prior to detection. Light propagating through tissue is attenuated by a factor of between 3 and 300 per centimeter depth, depending on the wavelength of light and type of tissue. Blue light is attenuated more than red, which causes spectral distortion. In principle, if the absorbance spectrum of all tissue components is known and the acquired signal is sufficiently intense, one can estimate the concentration of each component and deconvolve the original source spectrum based upon the acquired spectrum. Reconstruction can be simpler and more robust if a nearby spectral reference region (e.g. a region with uncoated phosphor or a region with a different phosphor) is used to measure attenuation in the tissue. In one embodiment, the surface of an implantable device incorporating the system can be patterned with discrete reference regions, which can take advantage of the high spatial resolution available by the method, to acquire reference spectra. Alternatively, detection of the optically detectable response of the chemical indicating element can be based on phosphorescent lifetimes, which are unaffected by tissue scattering or absorption. Many indicator dyes (e.g., many oxygen indicator dyes) have long phosphorescent lifetimes that are modulated by oxygen quenching. In such an embodiment, a fast-decaying scintillator can be utilized in conjunction with a chemical indicating element that has a long phosphorescent lifetime so as to distinguish the lifetime of the dye from that of the phosphor.

The imaging technique for the system can measure local chemical concentrations in tissues at a depth of about 1 mm or greater with a resolution dicted by the X-ray beam width. Beneficially, it is the X-ray beam width, not the visible photons, that limits the spatial resolution of the chemical images. According to one embodiment, a collimated X-ray beam (e.g., a 100 μm collimated beam) can be used to excite the scintillator. The optical emission of the system can then be measured at two wavelength bands to determine concentration of the targeted analyte. The image can be created by scanning the sample or the beam (in a scanning disk configuration) with a resolution limited by the beam width.

FIG. 2 schematically illustrates an excitation and detection system as may be utilized in conjunction with the sensing system. A narrow collimated beam 10 from an X-ray source 14 can be scanned through a tissue 12. Luminescence from the sensing system can be collected and measured, for instance by use of a photodetector 16. In one embodiment, the luminescence can be collected at two frequencies using a dichroic image splitter 18.

The size of the X-ray dose can be varied as needed to provide a good image. The detected signal will be proportional to incident X-ray flux, so there is a natural trade-off between X-ray dose and signal to noise ratio (S/N). For a given imaging area, there is also a tradeoff between image resolution (number of pixels) and S/N per pixel. A typical cancer radiotherapy regime involves a series of 1-3 Gy/day up to a total of 30-70 Gy (1 Gy=1 J of absorbed radiation/kg tissue). Although large ionizing doses are known to cause cancer the, effect of whole body doses of less than about 100 mSv on the incidence of cancer is still controversial and indeed some studies show that low levels of radiation enhance immunity and decrease cancer incidence. In general, an X-ray dose of less than about 20 mGy, less than about 10 mGy, or less than about 5 mGy can be sufficient for excitation of the scintillator. For example, doses of about 1 mGy can be sufficient for chemical imaging in one embodiment. The dose is also localized to the scanned region which further reduces the average whole body dose.

An approximate model will now be described for estimating how many visible photons are collected for 1 mGy of radiation. 50 keV X-ray energy is assumed, which provides optimal contrast compared to tissue; X-ray attenuation is neglected for simplicity (1/e depth is ˜5 cm at this wavelength).

The number of visible photons detected, nᵥᵢₛ_{_{—}}_{det}, is equal to the number of X-ray photons absorbed by the beacons, n_{X}_{_{—}}_{abs}_{_{—}}_{b}, times the conversion efficiency to visible light, Q_{X}_{_{—}}ᵥᵢₛ, the FRET efficiency from a lanthanide center to a gold indictor dye, Q_{FRET}, times the transmittance through the tissue Tₜ time the optical collection efficiency Q_{coll} times the quantum efficiency of the photodector Q_{det}. incident upon each nanoparticle, n_{X}_{_{—}}_{inc}, times the cross-section of each nanoparticle, σ_{beacon}, times the number of nanoparticles in the beam:

nᵥᵢₛ_{_{—}}_{det}=n_{X-abs}_{_{—}}_{b}*Q_{X-vis}*Q_{FRET}*Tₜ*Q_{coll}*Q_{det}. [Equation 1]

Estimating
Q_{X-vis}=60,000 photons/MeV=3,000 at 50 keV,
Tₜ=0.01, Q_{coll}=3% (15° degree acceptance angle), and
Q_{det}=50%, it is thus determined:

nᵥᵢₛ_{_{—}}_{det}=n_{X-abs}_{_{—}}_{b}*1. [Equation 2]

Thus, every X-ray photon absorbed by a beacon produces a detected photoelectron. At 50 keV, LuI₃ nanoscintillators have an X-ray attenuation coefficient approximately 500 times greater than an equal volume of soft tissue. A 1 mGy X-ray dose (1.25×10¹¹ X-rays/kg tissue) with an average volume ratio (volume scintillator/volume tissue) of 10⁻¹¹ will collect approximately 500 X-rays, and emit 150,000 photons, of which 500 will be detected as photoelectrons. Although the model is only approximate within 1-2 orders of magnitude, the model suggests an intense signal is expected with relatively dilute concentrations of nano-sized scintillator particles.

The model also suggests that the most effective methods of increasing visible light output given constant X-ray intensity will be improving the nanoparticle quantum efficiency, cross-section, concentration, and using a larger numerical aperture optical system with more detectors. Use of an integrating hemisphere will increase the amount of light captured although some light will be lost to tissue absorption. Optical fiber coupling may be used to increase the distance of the photodetector from stray backscattered X-rays.

The X-ray induced visible light can be detected at two visible wavelengths using a dichroic image splitter, and the X-ray beam can then be scanned in order to construct a high resolution chemical image. The acquired image can have a resolution limited by the scanning X-ray excitation beam width and can provide chemical specificity from the chemical indicating element.

The sensing systems can be utilized in a wide variety of chemical sensing applications. For example, the systems can be utilized in in vitro, ex vivo, and in vivo sensing applications. In one embodiment, a sensing system can be utilized in conjunction with portable X-ray sources and photodetectors so as to be utilized as a field-portable device. In another embodiment, images can be co-registered with X-ray transmission imaging and optical microscopy on the same device to provide additional information about the local area being examined.

Beneficially, the sensing system can be located on/in an implantable device so as to provide very specific information. For instance, in one embodiment, the sensing system can be designed to detect local chemical changes on an implant surface. Such surface sensitivity can provide medical imaging not previously available, and can be utilized to detect specific conditions particular to implant surfaces, such as detection of biofilm chemistry and biofilm migration.

Biofilms display heterogeneous pH and oxygen concentrations with strong variations over the 50 μm size scale. This environmental heterogeneity is a key reason for biofilm antibiotic resistance, but also provides an opportunity for detection provided that sufficient optical resolution as is capable in the disclosed sensing systems can be utilized. The spatial resolution of disclosed sensing systems can allow multiple analytes to be detected in different regions of an implantable device on a very small scale. Moreover, lifetime-based sensing schemes allow measurements independent of spectral distortion.

The disclosed sensing system has a wide range of applications of interest from fundamental tissue and animal biochemistry research to biomedical applications. While the detection and imaging of heterogeneous biofilm products on the surface of implanted medical devices is one particular application for disclosed systems, other applications, such as determination of localized hypoxia and/or acidosis in cancer treatment protocols are likewise encompassed herein.

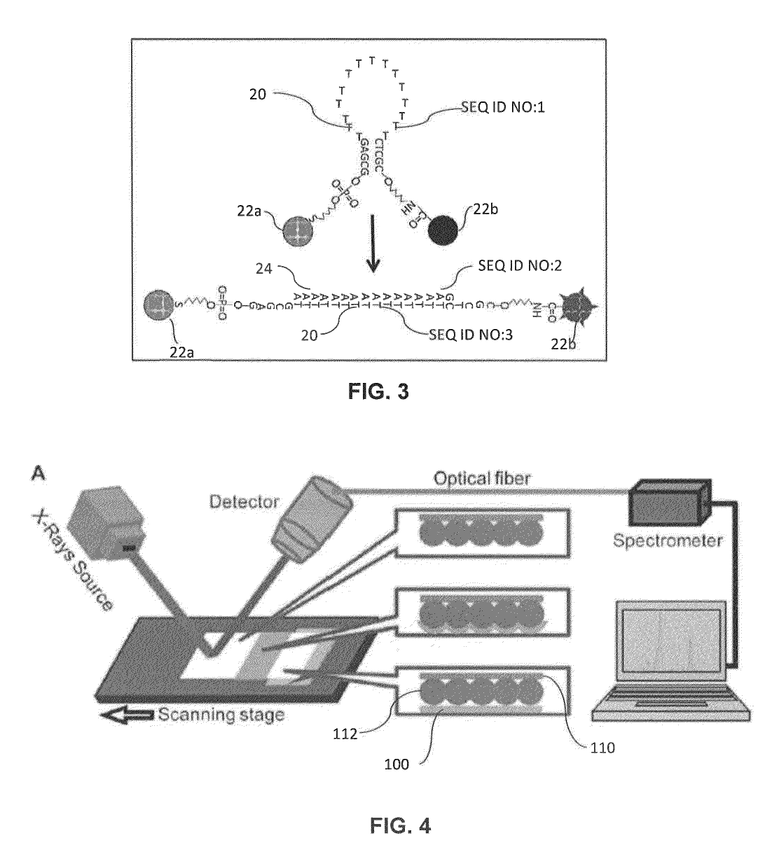

Another sensing system and illustrated in FIG. 3 is a molecular beacon that includes a polynucleotide, e.g., a DNA molecule 20 (SEQ ID NO: 1) in conjunction with a FRET donor/acceptor pair 22a/22b. The molecular beacon can switch its luminescence intensity upon hybridization with a complimentary single stranded DNA sequence 24 (SEQ ID NO: 2/SEQ ID NO: 3). The switching characteristic of the sensing system as designated by the directional arrow in FIG. 3 is due to changes in DNA conformation and resultant changes in FRET between a luminescent source 22a and quencher 22b.

In one embodiment, the molecular beacon can utilize an X-ray scintillator as the donor 22a and a gold nanoparticle as the acceptor 22b. In the normal closed configuration, X-ray excitation is transferred to the gold nanoparticle and luminescence is quenched by the gold due to Landau damping. When the beacon binds to a complimentary DNA sequence, however, the distance between the scintillator and the chemical indicating element increases, and the scintillator luminesces without quenching from the gold chemical indicating element. Over 99% quenching efficiency can be obtained in one embodiment due to the long lifetime of the scintillator, e.g., Ru and Eu ions. In one embodiment, luminescence can be enhanced due to increased local electromagnetic fields within one radius of the acceptor 22b, provided it is not sufficiently close (e.g., less than about 10 nm) to cause FRET quenching.

The molecular beacon illustrated in FIG. 3 is representative of a broad class of conformation-based sensing systems including those using a wide range of DNA aptamers and proteins, such as calcium sensitive calmodulin, as well as proteases that cleave a protein linking the quencher to the scintillator increasing the scintillators luminescence intensity and lifetime, as shown schematically in FIG. 3. A DNA molecular beacon can be relatively simple to modify according to standard methodology as is known in the art (e.g. by changing the length of the loop structure or creating mismatches in the complimentary sequence). For example, the spacing between the donor/acceptor pair can be modified to optimize fluorescence in the open configuration while maximizing quenching in the closed configuration.

An Aldrich synthesis method as is known in the art can be utilized to form a molecular beacon. For instance, a thiol group can be adsorbed to a gold nanoparticle surface as well as an amine group to conjugate to the nanoscintillator through EDC coupling. The nanoscintillators can then be functionalized with mercaptoundecanoic acid for conjugation to DNA.

Another sensing system encompassed herein includes micro- or nano-sized particles coated with an opaque hemispherical half-shell of metal. The metal coating material of the particles blocks fluorescence excitation and emission from the coated hemisphere. The particles can include a magnetic material such that they rotate and blink in response to external rotating magnetic fields. This blinking signal can be separated from background signals, e.g., autofluorescent background signals, thus enabling detection of local viscosity and drag based on the blinking rate.

In one embodiment, the particles can be loaded with indicator dyes and labels for no-wash spectrochemical sensing in autofluorescent samples. For instance, the particles can be utilized to study the mechanics of intracellular rotational transport and phagocytosis rates. Rotation rate and viscosity can be detected through tissue with autofluorescence about 2,000 times more intense than the fluorescence of the particles. Using X-ray excitable materials on the particles in conjunction with a scanning X-ray beam can provide higher spatial resolution sensing, for instance to measure phagocytosis in mouse lungs.

The present disclosure may be better understood with reference to the Examples, provided below.

### Example 1

### Reagents and Solutions

Hydrogen peroxide aqueous solution (30% w/w) was purchased from BDH Chemicals Ltd (Poole, Dorset, UK). Europium doped Gadolinium oxysulfide (Gd₂O₂S:Eu) was purchased from Scintillator Technology Ltd. (Stevenage, UK) and contained microparticles that ranged in size from about 2 to about 15 μm with a nominal diameter of 8 μm. Silver wire (>99.99%, 1 mm diam.) and gold wire (99.999%, 1 mm diam.) were purchased from Sigma-Aldrich (St Louis, Mo., USA). Double sided tape (3M 666, 1×1296 Inch) was purchased from 3M Company (St. Paul, Minn., USA). Cover glass (No. 0, 24×60 mm) was purchased from Electron Microscopy Sciences (Fort Washington, Pa., USA). Immersion oil was purchased from Leica Microsystems (Wetzlar, Germany). Deionized (DI) water was purchased from EMD Chemicals Inc. (Gibbstown, N.J., USA). Pork was purchased from Ingles Market, Inc. (Asheville, N.C., USA). Reynolds Wrap Quality Aluminum Foil was purchased from Consumer Products Division of Reynolds Metal (Richmond, Va., USA). All chemicals were used as received without further purification.

### Instrumentation

An X-ray diffractometer (Rigaku; MiniFlex, Cu—Ka) was used as an X-ray source. For X-ray scintillator luminescence, the X-ray diffractometer was operated with tube voltage of 30 kV and tube current of 15 mA. The sample was mounted on a stepper motor stage (MTS 50, Thorlabs, Inc., Newton, N.J., USA) which was controlled by a program written in Labview (National Instruments, Austin, Tex., USA). To measure lifetime, the X-ray irradiation was modulated by an optical beam chopper equipped with a 1 mm thick aluminium chopper wheel (Stanford Research Systems, model SR540, CA). The intensity of the emitted light was measured with a photomultiplier tube (PMT) (R955, Hamamatsu Photonics, Japan) connected to Tektronix TDX 2004B Oscilloscope. The signal acquisition was triggered off of the chopper wheel photodiode. Data was transferred from the oscilloscope using an interactive measurement software v1.2 (NI SignalExpress Tektronix edition). The PMT was placed over a 0.5 cm thick piece of glass to block the small amount of scattered X-ray photons while passing the visible luminescence. Scanning electron microscopy (SEM) was performed on a SU6600 microscope operated at 20 kV. Luminescent and extinction spectra were acquired at room temperature with a FPC-400-0.22-1.5-UV fiber (Thor Labs) coupled photodiode array spectrometer (BRC741E-02 BWTEK Inc, Newark, Del., USA). Luminescent images were taken with a Nikon D90 digital camera with a 67 mm diameter lens at 50 mm FL and a macro lens adaptor. To increase the captured luminescence during imaging through tissue, a 3×16 cm piece of aluminum foil was placed beneath the scintillator film. All the parameters of the camera were controlled by the software of Camera Control Pro2 (Nikon Instruments Inc., Melville, N.Y., USA). All images were analyzed using Matlab R2009b.

Preparation of Gd₂O₂S:Eu Phosphor Film Coated with Silver and Gold Island Films

A 24×60 mm cover glass was covered with double sided tape. A single layer of particles was attached to the surface of doublesided tape by spreading 0.5 g europium doped gadolinium oxysulfide (Gd₂O₂S:Eu) powder on top of the tape. Excess particles were removed by rinsing in DI water, followed by drying at room temperature for 2 h. The sample was plasma etched for 5 min in a Harrick plasma etcher at media power in air plasma to make the surface hydrophilic. Finally the sample was coated with silver or gold using thermal vapor deposition.

### Silver and Gold Island Film Coating and Silver Dissolution in Hydrogen Peroxide Solution

The Gd₂O₂S:Eu film was coated with a thin layer of gold or silver. The metal was deposited using thermal vapor deposition (Auto 360 vacuum coater/thermal evaporator, Edwards, West Sussex, UK) under high vacuum (less than 5×10⁻⁶ Torr). The thickness of the metal was controlled using a shutter and measured with a quartz crystal microbalance. In order to study the rate of silver dissolution in H₂O₂, a Gd₂O₂S:Eu film was prepared with a 5 nm thick average coating of silver. The film was then incubated in 1 mM H₂O₂ and the sample was taken out for luminescent spectra in every ten minutes. In order to selectively dissolve only a portion of silver in H₂O₂, a 5 nm silver island film was masked with two strips of tape leaving an unmasked region between the two strips. The sample was then incubated in 1 mM H₂O₂ for 3 h to dissolve the unmasked region.

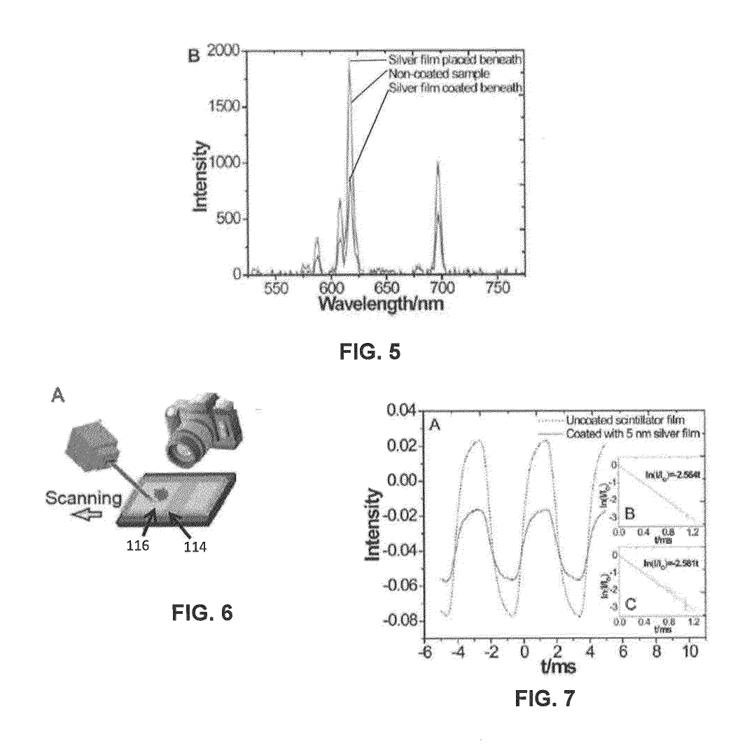

The silver coated scintillator film was irradiated with X-rays, and the luminescence intensity was measured. FIG. 4 schematically illustrates the testing system including the silver layer 100 the Gd₂O₂S:Eu 112, and the cover glass 110. As a control for whether silver contact was needed to attenuate the luminescence, a glass slide coated with 5 nm of silver island film was also placed beneath the scintillator. FIG. 5 illustrates the scanning results. The intensity of the silver-coated region was attenuated by 51% (at 617 nm) compared to the uncoated region. Direct coating of the silver onto the scintillators was required: a control prepared by placing a 5 nm silver-coated cover glass beneath the same slide of uncoated sample resulted in an increase of intensity (5%) rather than attenuation because of increased reflection and back scattering (see FIG. 5).

The multiple narrow and intense spectral luminescence peaks of Gd₂O₂S:Eu are advantageous because they are easily distinguished from tissue bioluminescence. The strong red and near infrared luminescence peaks are only weakly absorbed by tissue, although scattering greatly reduces resolution.

To measure the spatial resolution of a detection system, the system schematically illustrated in FIG. 6 was used. A rectangular X-ray beam (1.7 mm×10 mm) irradiated the silver coated region 114 of a scintillator sample 116 through 1 cm of pork tissue. The luminescent intensity was imaged with a Nikon D90 digital camera as the sample position was scanned relative to a fixed X-ray beam. Although each optical image showed a blurred 10 mm luminescence region due to scattering, the intensity and spectrum was modulated by local optical absorption. The total red, green, and blue pixel intensity in each picture was analyzed in Matlab. The total intensity collected as a function of position shows a clear knife-edge profile with a full-width of just 1.7 mm through 10 mm of tissue (FIG. 7), limited by the X-ray beam width. The slight broadening from 1.6 mm without the tissue is likely due to misalignment of the scintillator film after placed under 1 cm pork, or a halo effect from scattering of soft X-rays. FIG. 7 at A illustrates the luminescence responses resulting from X-ray pulse at 250 Hz as measured with a photomultiplier tube (FIG. 7 at B). FIG. 7 at C shows a plot of the log of the intensity vs. time after the chopper blocked the X-ray beam. Digitization noise is evident at low intensities. Lifetimes for coated and uncoated samples were 0.387 ms, and 0.390 ms, respectively.

### Luminescence Lifetime

To determine if the silver caused dynamic quenching of the X-ray luminescence, luminescence lifetime was measured. An optical chopper was placed in front of the X-ray beam and rotated at 250 Hz. A small amount (about 3%) of X-ray energy passed through the 1 mm thick Al chopper wheel even in the blocked state; however, a clear signal modulation was evident (see FIG. 7). The luminescence lifetime was determined from the shape of the curve. After a short transition period while the chopper shifts from not blocking the X-ray beam to fully blocking the beam, the intensity is expected to decay exponentially as follows:

I(t)=I₀e^{-t/τ}

where I(t) is the intensity at time, t, I₀ is the intensity at time t=0,and τ is the luminescence lifetime of the X-ray scintillator.

A plot of In(I) vs. t shows a linear curve indicating a single lifetime over the observed timescale (FIG. 7 at B, C). The lifetime of scintillator luminescence from silver coated and uncoated samples is 0.387 ms and 0.390 ms respectively. The lifetimes are similar to the nominal value quoted for the Gd₂O₂S:Eu scintillator of no more than 0.45 ms. The unchanged lifetime between coated and uncoated samples indicate that dynamic quenching was not responsible for attenuated luminescence intensity, and absorption is the most likely alternative.

### Effect of Silver Film Thickness on Luminescent Absorption

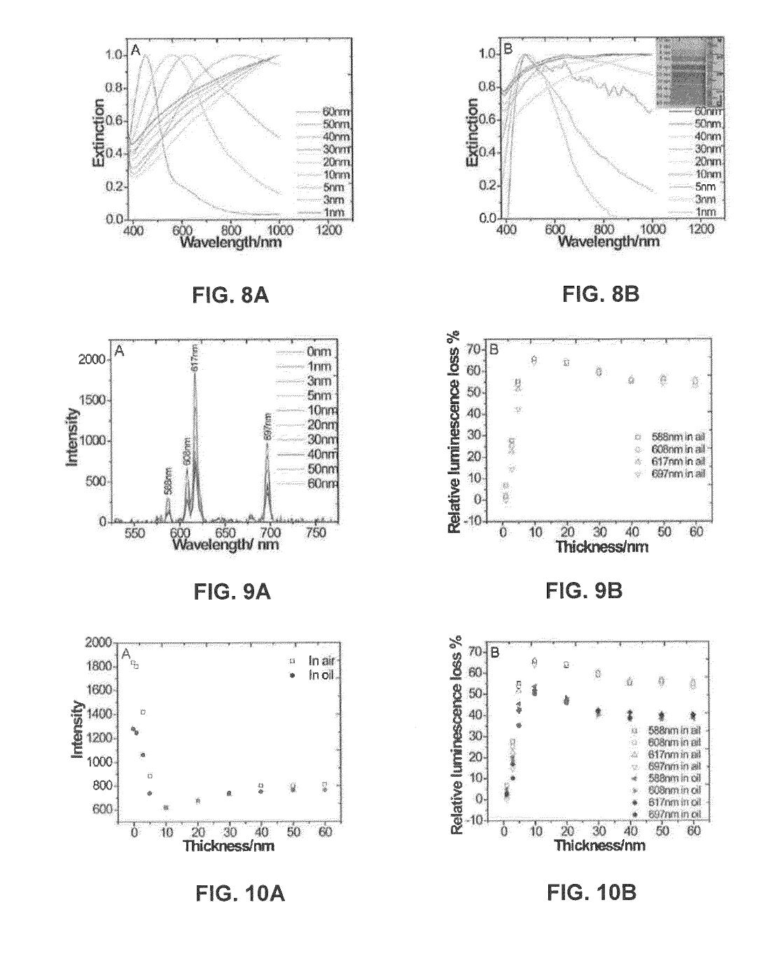

To determine if absorption was responsible for the reduced luminescence intensity, intensity was measured at various wavelengths as a function of silver film thickness. It was reasoned that if absorption were responsible, the signal would be smaller at wavelengths which are more strongly absorbed by silver nanoparticles. The LSPR absorption spectrum depends strongly on nanoparticle shape. Silver island film nanoparticles are formed during vapor deposition, and as more silver is deposited the particles become flatter until a continuous film is formed. As progressively thicker silver films were deposited, the particles change in shape from small and approximately spherical to larger flatter particle shapes and eventually continuous films with some surface roughness. For particles deposited upon a flat glass surface, this change in shape leads to a larger transverse LSPR resonance and a red-shift in extinction spectrum as shown in FIG. 8A. When the silver is deposited upon the angled and faceted surfaces of the scintillator microparticles the extinction spectrum (FIG. 8B) is blue-shifted compared with the spectrum on flat glass due to changes in nanoparticle orientation and shape.

The luminescent spectrum was shown to depend on the silver deposition thickness (see FIG. 9A and FIG. 9B). The variation of relative luminescence lost for film from 1 nm to 10 nm can be explained by the size and shape-dependent absorption properties of the nanoparticles in the silver island film. (See FIG. 8A). In general, the luminescent attenuation order for each peak is 588 nm>608 nm>617 nm>697 nm which is consistent with the extinction spectra of silver film on scintillator particles (FIG. 8B). For films thicker than 10 nm (more than the optical penetration depth of silver), the silver largely behaved as a glossy mirror, and the attenuation effect saturates.

The above experiments indicated that absorption was the most likely explanation for the silver attenuation because the attenuation showed a spectra dependence that was consistent with the silver nanoparticle extinction spectrum. However, since the silver coating was beneath the scintillators, and the camera and spectrometer detected light emitted through the top of the particles, the silver must absorb light that would otherwise be reflected from the bottom of the particles (e.g. due to refractive index mismatches). The refractive index of Gd₂O₂S:Eu is between 2.1 and 2.3 RIU. Assuming an intermediate refractive index of 2.2, the scintillator/air interface has a critical angle of 27° that corresponds to reflection from a solid angle of 1.82π², or 91% of an incident angles. In addition, 14% of normally incident light reflected at the Gd₂O₂S/air interface. For comparison, the critical angle for the scintillator/double-sided sticky tape interface is 42°, assuming a refractive index of 1.5 for the tape, this corresponds to reflection from a solid angle of 1.54πE² or 77% of incident angles; the normal reflection coefficient is 3.6%. Therefore, most of the light is reflected multiple times in the scintillator and most of light escapes through the top of scintillator/tape interface. This hypothesis explains why a silver coating below affects the light above. It also explains why placing a 5 nm silver island film below the scintillators increases the luminescence but by only 5% (see FIG. 5). To further test this hypothesis, the scintillator film was incubated in microscope immersion oil with a refractive index of 1.518 RIU (see FIG. 10A, 10B). The overall intensity of light collected through the top of the film decreased, consistent with a decrease in reflection from the bottom surface (FIG. 10A). In addition, the relative effect of attenuation from silver absorption was less significant after adding oil which is consistent with a reduced amount of internal reflection (FIG. 10B). These results support the internal reflection hypothesis. Internal reflection increases the effective absorption path length and enhances local absorption from nanoparticles and dyes on the surface which is adventurous for measuring small changes in absorbance. Furthermore, the particles can be designed to detect changes in local refractive index, a feature which can be employed to observe label-free protein binding and polymer degradation.

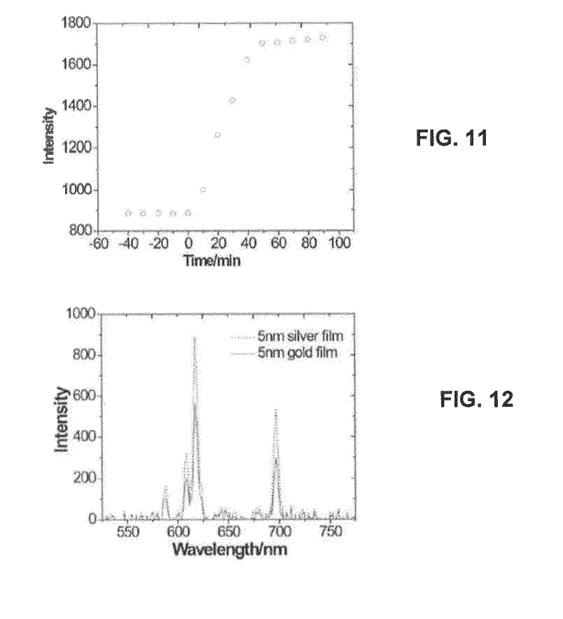

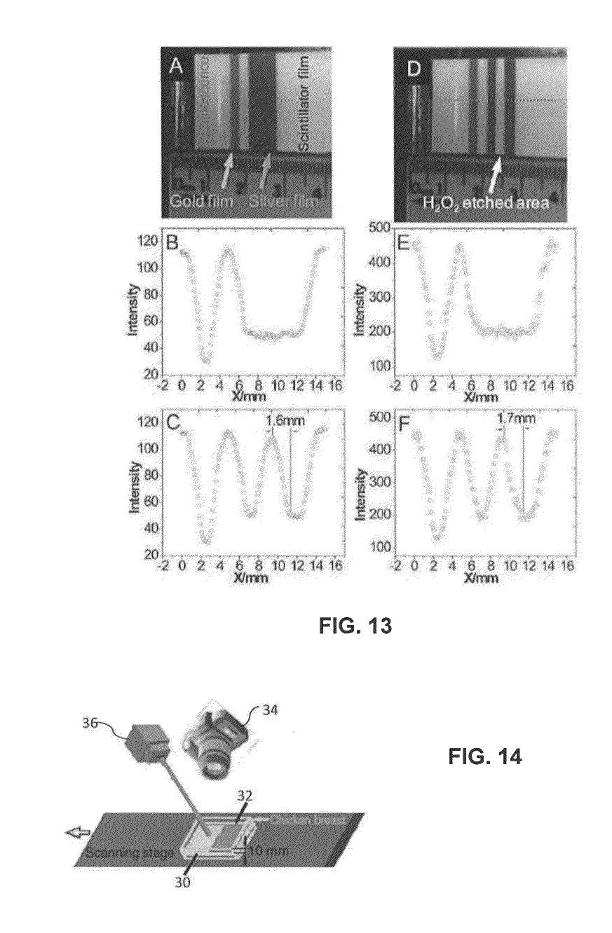

To study the dissolution of a silver island film in H₂O₂, 5 nm of silver was vapor deposited upon a Gd₂O₂S:Eu scintillator film, and the luminescent spectra were recorded in time during dissolution in a 1 mM H₂O₂ aqueous solution. The dissolution of the silver was indicated by the increase in luminescent intensity. As shown in FIG. 11, after the silver island film dissolved in H₂O₂, the luminescent intensity recovered to normal intensity within experimental error from sample to sample variation. Although 1 mM H₂O₂ concentration is a very high level for physiological conditions, the dissolution was relatively rapid (30 min), compared to a few weeks in physiological conditions. For example, assuming that rate is proportional to concentration, 30 min at 1 mM corresponds to a 7 mM concentration dissolving in 3 days or 700 nM over one month. In addition, the physiological rate may be different due to changes in pH, increased temperature (37° C. instead of 23° C.), extracellular components including chloride ions and metal binding enzymes, adsorption of protective proteins, and mechanical abrasion. The scanning technique is uniquely suited to study silver dissolution in situ. Silver dissolution can be studied over different time periods by tuning the thickness of silver island film. To detect low concentrations of H₂O₂ (1 mM) in thick tissue with high spatial resolution, a narrow X-ray beam (1.7 mm×10 mm) can be used to excite the scintillator film. In these experiments, a 5 nm thick silver film coated on Gd₂O₂S:Eu was selected since the luminescent reduction was enough to distinguish (FIG. 10A) and the small particle size reduced the time required to dissolve the silver (FIG. 11). The silver coated scintillator film was incubated in 1 mM H₂O₂ solution 100 ml of 30% (w/w %) H₂O₂ solution was added in 1000 ml DI water (pH 5.5)) at room temperature for 3 h. A 5 nm thick strip of gold was also deposited as a chemically inert control. FIG. 12 shows that the 5 nm gold film reduces the intensity more than the 5 nm silver film. This intensity difference and the stability of gold in physiological conditions make the gold film a good reference and fiduciary marker. In order to image local changes in silver absorbance, some regions of a silver film were masked with double sided sticky tape prior to exposure to 1 mM H₂O₂. FIG. 13 at A is a photograph of a scintillator film coated by a strip of silver 5 nm thick and 7.4 mm wide as well as a strip of vapor-deposited gold, 5 nm thick and 2.3 mm wide. The silver film was masked with two thin strips of tape so that only a section in the center was unprotected. The sample was plasma etched for 5 min in air plasma to make all regions hydrophilic. The sample was then incubated in 1 mM H₂O₂ for 3 h to dissolve the unprotected section of silver film (See FIG. 13). A one dimensional image was acquired from the film before and after silver dissolution, and the luminescence was performed with and without placing a 1 cm thick section of pork tissue above the film. The H₂O₂ etched area can be clearly identified by the increased luminescence, while it can be distinguished from the gold coated region with lower luminescence intensity. The gold coated region was unaffected by H₂O₂ providing a control strip. Most importantly, the resolution of the scan was hardly affected by the 1 cm of pork demonstrating high resolution imaging through thick tissue.

### Example 2

### Materials

Methyl Red (C₁₅H₁₅N₃O₂) and filter paper (medium porosity) were purchased from Fisher Scientific Co. (Fair Lawn, N.J., USA). pH buffers solution was purchased from BDM Chemicals Ltd (Poole, Dorset, UK). Terbium-doped gadolinium oxysulfide scintillator powder (Gd₂O₂S:Tb) and Europium doped Gadolinium oxysulfide (Gd₂O₂S:Eu) were purchased from Phosphor Technology Ltd. (Stevenage, UK). Both Gd₂O₂S samples contained microparticles that ranged in size from 2-15 μm with a nominal diameter of 8 μm. Carboxymethyl cellulose sodium was purchased from TCI (Tokyo, Japan). Cover glass was purchased from Electron Microscopy Sciences (Fort Washington, Pa., USA). Deionized (DI) water and ethanol were purchased from EMD Chemicals Inc. (Gibbstown, N.J., USA). Chicken breast was purchased from Coleman Natural Foods, Inc. (Golden, Colo., USA). All chemicals were used as received without further purification.

### Experimental Procedure

Preparation of scintillator film: 1.0 g Gd₂O₂S:Tb scintillator powder was mixed with 2 mL carboxymethyl cellulose sodium aqueous solution (0.5% w/v) and the entire solution was applied to a cover glass (24×60 mm) and allowed to dry overnight. A film of Gd₂O₂S:Tb and Gd₂O₂S:Eu scintillator coated on the same glass slide (25×75 mm) was prepared in a similar way. 0.5 g Gd₂O₂S:Tb scintillator and 0.5 g Gd₂O₂S:Eu scintillator were each drop-coated onto a separate cover glass (24×60 mm). These two scintillator-coated cover glasses were brought together and stuck to a glass microscope slide (25×75 mm) using double-sided tape.

Preparation of Methyl Red Dyed Filter Paper and pH Measurement:

A 12×100 mm strip was prepared from filter paper of medium porosity and this strip was immersed in 600 μL ethanolic solution of Methyl red (0.05% w/v) over night and dried thoroughly. The ethanolic solution of methyl red (0.05% w/v) was prepared by adding 0.0010 g methyl red powder into 2.00 ml ethanol. The 12×100 mm methyl red dyed strip then was cut into a series of 12×8 mm pH-strips. Following, these pH-strips were placed onto a No. 0 coverslip covering the scintillator film. A series of calibrated pH buffer solution (pH 2.00-13.00) were then pipetted onto each pH strip (10 μL). The stage was moved so that each pH strip was irradiated and a series of five spectra were recorded from different areas of the strip to determine the average and standard deviation of the peak intensity ratio.

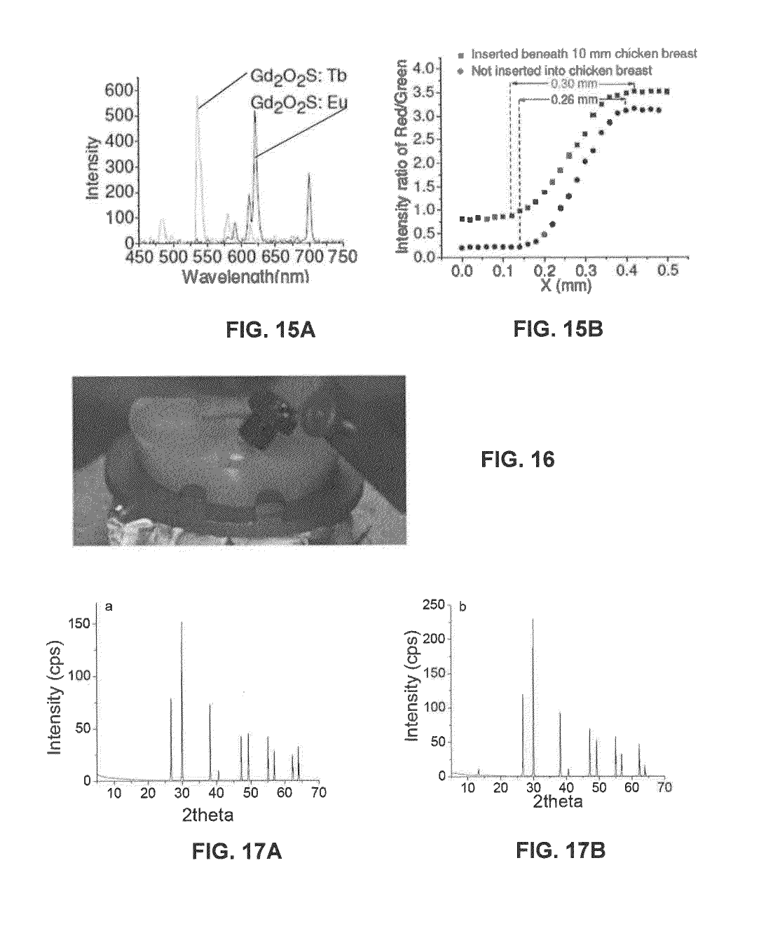

As illustrated in FIG. 14, the thin film of green phosphors (Gd₂O₂S:Tb) 30 and red phosphors (Gd₂O₂S:Eu) 32 (Phosphor Technologies Inc.) was fabricated with a sharp interface between the two. The color of the light was imaged with a Nikon D90 digital camera 34 as the sample position was scanned relative to a fixed X-ray beam 36. The scintillators were then sandwiched between two 1 cm thick sections of chicken breast and the experiment was repeated. Although the optical image blurred to 8 mm, the color indicated which region of the phosphor was under excitation. The total red, green, and blue pixel intensity in each picture was analyzed in Matlab. FIG. 15A shows the spectrum of red and green phosphors and FIG. 15B shows that the resolution for R/G intensity through 1 cm of tissue was just 300 μm, limited by the X-ray beam width (the slight broadening from 260 μm without the chicken is likely due to slight misalignment of the scintillator film after placing between the chicken pieces). FIG. 16 is a photograph of a polyethylene knee implant with embedded green phosphor and pH paper.

To determine the phase of the samples, X-ray powder diffraction was performed at 40 kV and 40 mA using a Rigaku Ultima IV X-ray diffractometer with CuKα radiation at a scanning rate of 0.5°/min from 5° to 70°. The XRD patterns (FIGS. 17A, 17B) were almost identical. They displayed narrow peaks indicative of particles with average crystal domains >90 nm in diameter.

### Example 3

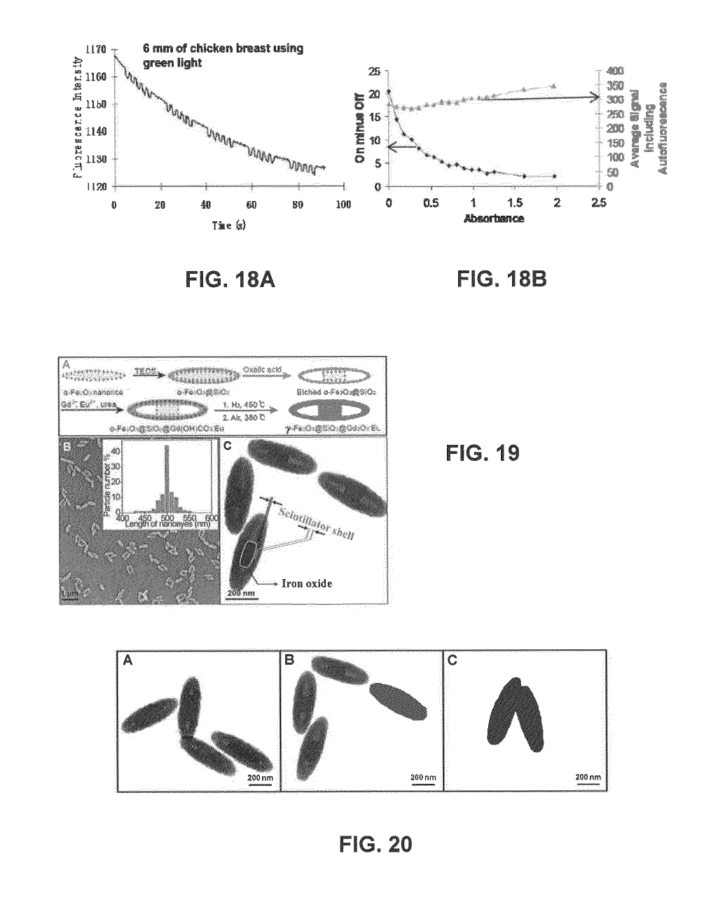

Magnetic/fluorescent polystyrene microspheres were deposited on a glass slide and coated with aluminum using thrermal vapor deposition. After magnetization, the particles were removed and suspended in water by sonication. A solution of 50/50 glycerol/water by weight with surfactant was mixed with the coated microspheres. This solution was prepared into a rectangular capillary, sealed, and placed in a Petri dish with 40 ml of water. Intralipid was added to the Petri dish in 60 microliter aliquots. A series of fluorescent spectra were taken at each Intralipid concentration. For each concentration, the fluorescence intensity was acquired in time to observe the modulated and unmodulated signal. Signal subtraction showed that the modulated signal decreased as the solution became more turbid, but could still be observed through a medium with an optical density of 2 (FIGS. 18A and 18B).

These sensors blink when they rotate in response to rotating external magnetic fields. This blinking signal can be separated from backgrounds allowing chemical sensing in highly autofluorescent media. In addition to chemical sensing based on fluorescence spectra, the rate of rotation provides information about local viscosity, elasticity and biomechanical torques. FIGS. 18A and 18B show fluorescence signal intensity from the particles embedded 6 mm into chicken breast, which intensely autofluoresces and also bleaches. The blinking rate measured the average local viscosity, and the fluorescence spectrum could be used to measure chemical concentration, but much of the intensity was reduced due to poor penetration of green excitation light. The intensity was reduced by a factor of 40, but is easily visible over the background.

### Example 4

### Materials

Iron (III) chloride anhydrous and tetraethoxysilane (TEOS) were purchased from Sigma-Aldrich (St. Louis, Mo., USA). Gadolinium Nitrate, Europium nitrate, potassium dihydrogen phosphate, and sulfur powder (99.5%) were purchased from obtained from Alfa Aesar (Ward Hill, Mass.). Ethanol (anhydrous, denatured), urea, oxalic acid, ammonium hydroxide, and nitric acid were obtained from BDH Chemicals Ltd (Poole, Dorset, UK). Deionized (DI) water was purchased from EMD Chemicals Inc. (Gibbstown, N.J., USA). Polyvinylpyrrolidone (PVP K-30, MW 40,000) was purchased from Spectrum Chemicals (Gardena, Calif.). Agarose (melting point 88±1° C.) was purchased from Shelton Scientific (Peosta, Iowa). All chemicals were used as received without further purification.

### Characterization Methods

Transmission and scanning electron microscopy (TEM) were performed on a H9500 operated at 300 kV and HD2000 microscope operated at 100 kV, respectively. An X-ray diffractometer (Rigaku; MiniFlex, Cu Kα) was used to characterize the XRD pattern of the magnetic scintillators. For fluorescence spectra, 480 nm light was used to excite the scintillators.

To measure radioluminescence, X-ray was generated by a mini X-ray tube (Amptek Inc. MA, USA), the X-ray tube was operated with tube voltage of 40 kV and tube current of 99 μA. The sample was mounted on a Leica Microscope (Leica DMI 5000M, Wetzlar, Germany) equipped with a DeltaNu DNS 300 spectrometer (Intevac-DeltaNu, Laramie, Wyo. USA) with a 150 lines/mm grating blazed at 500 nm.

The Zeta-potential of the nanoparticles was determined using a Malvern Instruments Zetasizer Nano ZS with a 633 nm He—Ne laser. Prior to the experiment, the particles were diluted in distilled water (0.1 mg/ml). Magnetization measurements were performed at the designated temperature using vibrating sample magnetometer (VSM) option of physical property measurement system (PPMS, Quantum Design, USA), with the applied magnetic field sweeping between +/−3.0 Tesla at a rate of 50 Oe/sec. Determination of the gadolinium and iron content in a sample was performed by inductively coupled plasma (ICP)—(Optima 3100 RL; Perkin-Elmer).

In order to magnetically modulate the optical scattering from the magnetic luminescent particles, the particles were oriented and rotated in a magnetic field. A permanent magnet (NdBFe, 1″ in diameter, 3″ in length, magnetized through its diameter) was attached to a stepper motor and controlled by motion control software (Si Programmer; Applied Motion Products, Watsonville, CV). Every 3 s, the permanent magnet was rotated 90°, first clockwise and then anticlockwise.

All MRI experiments were performed on a Varian 4.7T horizontal bore imaging system (Agilent Inc, Santa Clara, Calif.). Samples, contained in 5 mm NMR tubes, were placed in a 63 mm inner diameter quadrature RF coil for imaging. MRI gradient echo scout images were collected in all three imaging planes (axial, coronal, and sagittal) for subsequent image planning, with repetition time (TR)=100 ms, echo time (TE)=4 ms, number of slices=20, slice thickness=2, matrix size 128×128, field of view (FOV)=40 mmx40 mm, number of acquisitions (NEX)=2. Relaxivity measurements were then collected on a single 2 mm thick imaging slice, approximately perpendicular to the long axis of the NMR tubes. The single slice, with FOV=36 mmx36 mm, was imaged using a T₂-weighted multi-spin echo imaging sequence with TR=3000, NEX=10, echo spacing=4 ms, number of echoes=10, and 128×128 matrix. T₂*-weighted images were collected using a gradient echo imaging sequence with TR=500 ms, flip=20°, 128×128 matrix, NEX=10, and echo times=[1.5, 3, 4.5, 9, 15 ms]. Following data collection, images were analyzed using Matlab 2011a (The Mathworks, Inc., Natick, Mass.). Regions of interest (ROI's), encompassing approximately 70-80 voxels, were manually drawn in each sample, and the signals from those voxels averaged to obtain a mean signal for each sample. The same ROI was used to calculate the mean signal of the sample across all echo times.

### Synthesis of Spindle-Shaped Hematite Particle

The template core-shell synthesis process is presented in FIG. 19 at A. Initially, monodispersed spindle-shaped hematite nanoparticles with controllable aspect ratios were fabricated were prepared according to a method as is known in the art. 100 ml of aqueous solution containing 2.0×10⁻² M FeCl₃ and 4.0×10⁻⁴ M KH₂PO₄ were aged at 100° C. for 72 hours. The resulting precipitate was centrifuged and washed three times with water.

Synthesis of Spindle-Shaped Hematite Particle with Silica Shell

To obtain monodispersed hematite-silica core-shell nanoparticles, the PVP assisted coating method was used. The spindle-shaped hematite particles synthesized above were dispersed ultrasonically to a 80 ml solution containing PVP (0.6 g), water (6 ml), and ethanol (74 ml). The suspension was stirred using a magnetic stir bar at room temperature and a solution of TEOS (270 μl) in 20 ml ethanol was added, followed by 4 ml of ammonia hydroxide. After 3 h, the reaction mixture was precipitated by centrifuging at 4000 rpm for 16 min. The particles were washed three times with ethanol and centrifuged to collect the product.

### Synthesis of Spindle-Shaped Magnetic Scintillators

The products above were suspended in 180 ml distilled water with 1.8 g PVP and 11.34 g oxalic acid (0.5 M) incubated at 60° C. for various times to form particles in which outer portions of the core was etched away (nanoeyes), or hollow particles (hollow nanorice). Solid particles (solid nanorice) were not subjected to the etching process. The hematite partially dissolved particles were collected by centrifugation and rinsed with DI water twice. The obtained particles were re-suspended with 3 ml Gd(NO₃)₃, 1.5 ml Eu(NO₃)₃ (80 mM), and 1.8 g PVP in pure water to form 300 ml of solution. 18 g of urea was added to the solution and the solution was maintained at 80° C. for 90 min. The precursor of magnetic scintillator was collected by centrifugation. The precursor of spindle-shaped precursor was calcined in a furnace at 600° C. for 60 min. In order to convert the α-Fe₂O₃ to magnetic γ-Fe₂O₃, the product was then transferred to a tube furnace with a H₂/N₂ (5%/95%) flow at 450° C. for 3 h. After that, the sample was calcined in the tube furnace at 350° C. for 24 h.

### Preparing Nanocomposites for MR Imaging.

T₂ and T₂* MR measurements were acquired for the spindle-shaped γ-Fe₂O₃@SiO₂@Gd₂O₃:Eu particles at a series of concentrations (0.8 mg/ml, 0.4 mg/ml, 0.1 mg/ml, and 0.05 mg/ml). The particles were dispersed in 0.5% agarose gel at 80° C. and cooled to room temperature in NRM tubes to set the gel. The gel prevented settling and aggregation allowing MRI imaging several days after preparation.

### Cell Viability Test

MCF-7 breast cancer cells were seeded at a density of 10,000 cells/well in a 96-well plate. Cells were stored at 37° C. at 5% CO₂ and attached to the plate overnight. Nanoparticles were suspended in media, sonicated for 10 minutes to disperse, and diluted to 250, 100, 50, and 10 μg/ml. Media was removed from wells and fresh media or nanoparticle in media was added to each well. Five repeats were done for each concentration. Nanoparticles were incubated with cells overnight and the next day a Presto Blue assay (Life Technologies) was performed. Media was removed and 100 μl of a 1:9 ratio Presto Blue in culture media was added to each well. Cells were incubated at 37° C. and 5% CO₂ for 45 minutes. Fluorescent intensity was taken with a plate reader with an excitation wavelength of 560 nm and an emission wavelength of 590 nm. Fluorescent intensity for each concentration of nanoparticle was normalized as a percentage of the fluorescent intensity of the control cells. Percent viability averages were plotted with error bars of one standard deviation.

### Structure and Morphology of Magnetic Luminescent Nanoeves (γ-Fe₂O₃@SiO₂@Gd₂O₃:Eu)

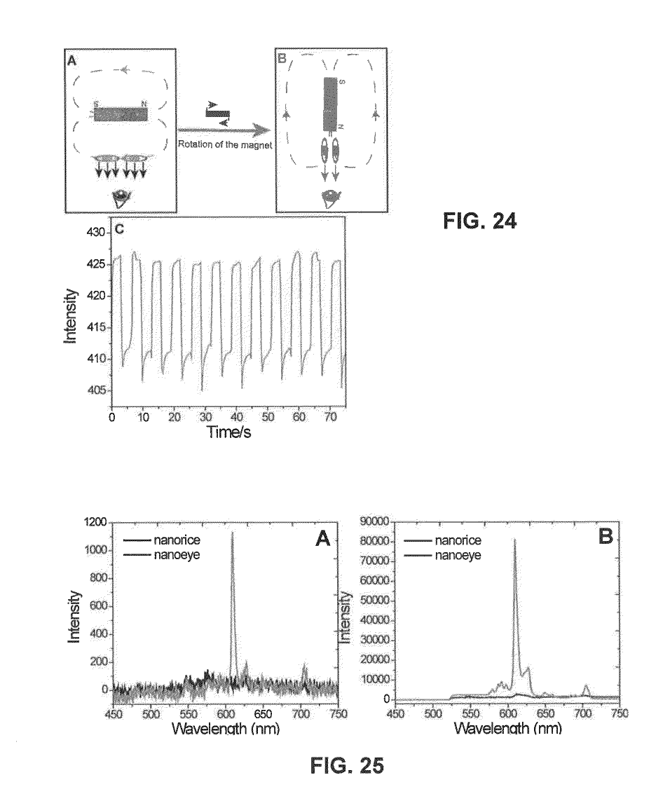

The particles were spindle shaped with an average length of 400 nm and diameter of 80 nm, however, by varying the synthesis conditions, the nanoparticle size can be tuned from about 120 to about 550 nm, and the aspect ratio from spheres to prolate spheroids. The colloidal hematite particles had a positive zeta potential of +26 mV which kept them well dispersed in solution due to electrostatic repulsion. FIG. 19 at B includes an SEM of the formed materials, the inset of FIG. 24 at B shows the size distribution of the formed nanoeyes, and FIG. 19 at C is a TEM image of the formed nanoeyes. After 9.5 h of etching, the magnetic cores of these nanoeyes were cylinder-shaped with an average length of 150 nm and diameter of 60 nm. In addition, the length of the iron oxide core was tunable from 0 to 400 nm with different etching times. For example, FIG. 20 illustrates TEM images of particles formed as described above but for difference in etching times. The particles of FIG. 20 at A were incubated in 0.5M oxalic acid at 60° C. for 8.5 hours, the particles of FIG. 20 at B were incubated of 9.5 hours, and the particles of FIG. 20 at C were incubated for 10 hours. The differences in length in the iron oxide cores can be seen in the images.

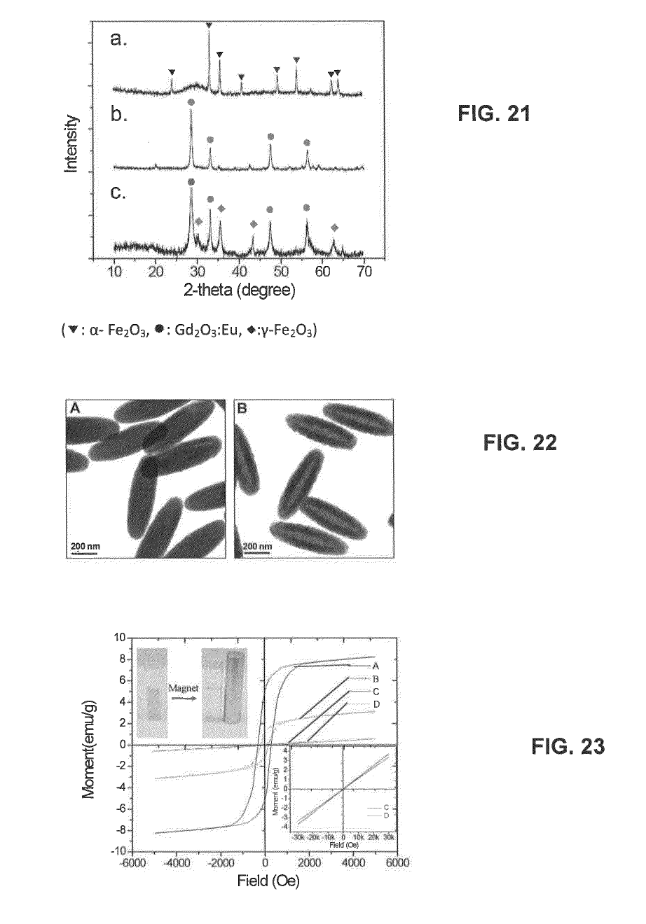

X-ray diffraction (XRD) was performed on the samples in order to investigate their structure and composition. FIG. 21 at a. shows the XRD pattern of the precursor nanoeyes (α-Fe₂O₃@SiO₂@Gd(OH)CO₃:Eu). The X-ray diffraction pattern of α-Fe₂O₃ (FIG. 21 at a.) is clearly distinguished from the broad peak at about 30° from the porous Gd(OH)CO₃:Eu. The XRD pattern of γ-Fe₂O₃@SiO₂@Gd₂O₃:Eu nanoeyes (iron oxide core was incubated in oxalic acid for 9.5 h) (FIG. 21 at b.) exhibits the characteristic diffraction peaks of cubic structure of Gd₂O₃. The γ-Fe₂O₃ core peaks in FIG. 21 at b. are indiscernibly weak because the cores have a small volume percentage and cross-section compared to the Gd₂O₃. However, the XRD pattern of γ-Fe₂O₃ can be readily distinguished under a thin shell (˜10 nm) of Gd₂O₃:Eu in the XRD pattern of particles in which the iron oxide core was incubated in oxalic acid for 9.5 hours with a thin Gd₂O₃:Eu shell. (FIG. 21 at c.).

### Magnetic and Optical and Properties of Magnetic Luminescent Nanoparticles

To elucidate the magnetic properties of the nanoeyes, similar shell structures were synthesized with solid iron oxide cores (solid nanorice) and hollow particles (hollow nanorice) with dissolved cores by incubation of the α-Fe₂O₃@SiO₂ in oxalic acid for 0 h and 17 h, (FIG. 22 at A and B, respectively). The solid-core nanorice (γ-Fe₂O₃@SiO₂@Gd₂O₃:Eu) and hollow nanorice (SiO2@Gd₂O₃:Eu) both were monodispersed with a radioluminescent shell shape almost identical to the nanoeyes.

The room temperature magnetic hysteresis loops of magnetic solid nanorice (A), nanoeyes (B), solid nanorice with hematite as the core (C), and hollow nanorice (D) are shown in FIG. 23. The hollow nanorice (SiO₂@Gd₂O₃:Eu) (FIG. 23 (D)) are paramagnetic, with minimal hysteresis and a magnetic susceptibility of 1.23×10⁻⁴ emu g-1 Oe-1 and showed no sign of saturation up to applied fields of 30 kOe. From the TEM images (FIG. 22) the core of the solid nanorice is approximately 13% of the volume and a similar percentage of the mass. The iron oxide was converted to maghemite (γ-Fe2O3) via H₂ reduction followed by oxidization in air (350° C. 24 h), and the particle color changed from red with the hematite core to brownish with the maghemite core. The solid nanorice magnetization curve (FIG. 23(A)) had two components, a hystertic ferrimagnetic (or ferromagnetic) component with a coercivity of 308 Oe and a saturation magnetization of ˜7.6 emu/g, and a paramagnetic component (linear slope at large applied fields). The saturation magnetization of the ferrimagnetic component is approximately 10% of the saturation magnetization of bulk γ-Fe₂O₃ (74 emu/g). These results are consistent with a core of about 10% maghemite by weight and a shell of about 90% by weight which is also consistent with the TEM images and ICP elemental analysis data. The hysteresis curve for nanoeyes (FIG. 23(B)) had a smaller ferrimagnetic component with a saturation magnetization of 2.3 emu/g, which is consistent with a 3-fold reduction in weight percent of maghemite. The coercivity decreased to 165 Oe likely due to the decreases in the aspect ratio of the iron oxide core. Although the saturation magnetization of the partially dissolved nanoeyes is smaller than the solid nanorice, the magnetization is still strong enough for rapid magnetophoretic separation (inset figure of FIG. 23).

The rotation of the magnetic nanoeyes in response to a rotating external magnetic field was demonstrated by measuring the scattering signal intensity as the particles rotated in response to a changing magnetic field. A solution of nanoeyes was placed in water/glycerol (v_{water}:v_{glycerol}=1:9) was placed on a glass slide and the scattering intensity was observed with a dark field microscope. The nanoeyes were orientated by an external magnetic field which rotated by 90° every 3 s. As depicted in FIG. 24, the scattering intensity was largest when the particles were oriented parallel to the sample plane and presented the largest scattering area. The scattering intensity decreased when the particles were oriented parallel to the optical axis. FIG. 24 at C shows the magnetically modulated optical scattering by the nanoeyes. This rotational modulation cannot be observed for spherical particles which have isotropic scattering properties.

In addition to magnetic rotation and separation, the nanoeyes displayed fluorescence under 480 nm excitation light (FIG. 25 at B) and radioluminescence under X-ray excitation (FIG. 25 at A). The main emission peak of the Gd₂O₃:Eu scintillator shell with cubic structure was observed at 610 nm, which corresponds to a red emission from the 5D₀→7F₂ Eu³⁺ transition. The fluorescence and radioluminescence of the solid core nanorice were hardly detected. The luminescence quenching is likely due to inner filter effects as the 10 nm silica shell spacer and hollow regions in the core would inhibit resonant energy transfer. The method of partially dissolving the iron oxide core reduced the quenching while maintaining the total nanoparticle size and volume. Carefully controlling dissolution time can allow optimization of the optical and magnetic properties. In addition, the hollow space that is formed around the iron oxide core in the nanoeyes can serve to encapsulate drugs for drug delivery applications as previously discussed.

The magnetic moment of the hollow nanorice obtained by completely dissolving the iron oxide was weak compared to the solid nanorice and nanoeyes, especially at low fields. As a result, they separated only very slowly with typical permanent magnet-generated magnetic fields and field gradients. The nanorice particles with a solid iron oxide core responded rapidly to magnetic fields but displayed only weak fluorescence and negligible radioluminescence. In addition, there is not much space within the core for dye and drug encapsulation. By partially dissolving the iron oxide core, nanoeyes were formed which display magnetophoresis and radioluminescence. They exhibited bright luminescence under UV (365 nm) and X-ray irradiation. Compared with the UV fluorescence, the X-ray luminescence of the nanoeyes provided a background-free image which can be used for deep tissue imaging. The hollow regions in the nanoeyes could also be used for indicator dye and drug encapsulation for theranostic applications. To test whether the pores in the Gd₂O₃ shell were large enough for small molecules to diffuse through, the nanoparticles were incubated in a solution of bromocresol blue dye, encapsulated the dye and particle with the roughly 10 nm silica layer, and washed and separated the particles via centrifugation. The dye could indeed be encapsulated as was evidenced by a change in color of the particles following incubation with the dye, while the solid core particles used as control could not encapsulate dye.

### Magnetic Luminescent Nano Articles as T₂ Contrast Agent

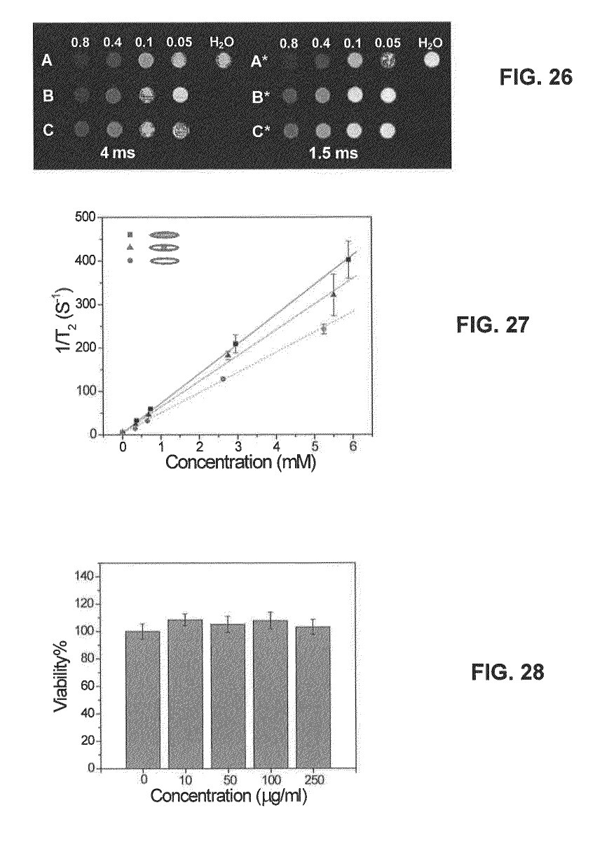

The magnetic radioluminescent particles described herein can serve as T₂ and T₂* contrast agents because of their strong magnetic moment in static MRI fields. FIG. 26 shows T₂ and T₂* weighted images after 4 ms and 1.5 ms, respectively of the solid nanorice (A and A*), the nanoeyes (B and B*), and the hollow nanorice (C and C*). Group A: T₂-weighted images of solid nanorice with concentration of 0.8 mg/ml, 0.4 mg/ml, 0.1 mg/ml, and 0.05 mg/ml. Group B: T₂-weighted images of nanoeyes with concentration of 0.8 mg/ml, 0.4 mg/ml, 0.1 mg/ml, and 0.05 mg/ml. Group C: T₂-weighted images of hollow nanorice with concentration of 0.8 mg/ml, 0.4 mg/ml, 0.1 mg/ml, and 0.05 mg/ml. Group A*: T₂-weighted images of solid nanorice with concentration of 0.8 mg/ml, 0.4 mg/ml, 0.1 mg/ml, and 0.05 mg/ml. Group B*: T₂*-weighted images of nanoeyes with concentration of 0.8 mg/ml, 0.4 mg/ml, 0.1 mg/ml, and 0.05 mg/ml. Group C*: T₂-weighted images of hollow nanorice with concentration of 0.8 mg/ml, 0.4 mg/ml, 0.1 mg/ml, and 0.05 mg/ml.

The decrease with echo time was fit to an exponential in order to calculate the relaxation rate, R₂=1/T₂ and R₂==1/T₂* at each concentration. These relaxation rates are shown as a function of concentration in FIG. 27. The curves are approximately linear, although there is some evidence of saturation at the highest concentration, 0.8 mg/ml. Fitting the points up to 0.4 mg/mL (2.6-3 mM), the relaxivities, r₂ and r₂* are 68.73 mM⁻¹ s⁻¹ and 274.11 mM⁻¹ s⁻¹ respectively for the solid nanorice; 58.10 mM⁻¹ s⁻¹ and 120.43 mM⁻¹ s⁻¹ for the nanoeyes, and 46.00 mM⁻¹ s⁻¹ and 111.76 mM⁻¹ s⁻¹ for the hollow nanorice. The relaxivity was calculated based on the total molar concentration of both Gd³⁺ and Fe³⁺. For all particles, r₂* was larger than r₂ because r₂ includes contributions from local static field inhomogeneities caused by the magnetic moment of the particles. The difference between r₂ and r₂* may provide more specificity towards the contrast agents, especially for the nanorice which display a factor of 4 increased relaxivity. The iron oxide core significantly increased the relaxivities, with the solid core providing the highest relaxivity and the hollow core the least.

### Nanoparticle Cytotoxicity Assay

In order to study the potential cytotoxicity of the rare earth elements in the magnetic imaging probes (solid nanorice, nanoeyes, and hollow nanorice), an in vitro cytotoxicity test of hollow nanorice was performed on MCF-7 breast cancer cells. Results are shown in FIG. 28. The viability of the untreated cells was set as 100% in order to calculate the viability of the cells treated by different concentration of X-ray scintillators. As can be seen, cell viability was not significantly affected by the Gd₂O₃:Eu nanoparticles up to concentration of at least 250 μg/ml (24 hr exposure).

### Example 5

### Materials

Tetraethoxysilane (TEOS), poly(styrenesulfonate sodium) (PSS, MW: ˜70,000), and iron (III) chloride anhydrous were purchased from Sigma-Aldrich (St. Louis, Mo.). Gadolinium nitrate, europium nitrate, and poly(allylamine hydrochloride) (PAH, MW: ˜15,000) were purchased from Alfa Aesar (Ward Hill, Mass.). Ethanol (96%), urea, oxalic acid, ammonium hydroxide, and nitric acid were obtained from BDH Chemicals Ltd. (Poole, Dorset, UK). Deionized (DI) water was purchased from EMD Chemicals Inc. (Gibbstown, N.J., USA). Polyvinylpyrrolidone (PVP K-30, MW 40,000) was purchased from Spectrum Chemicals (Gardena, Calif.). Agarose (melting point 88±1° C.) was purchased from Shelton Scientific (Peosta, Iowa). All chemicals were used as received without further purification.

### Preparation of Nanocapsules

Monodisperse spindle-shaped hematite nanotemplate particles with controllable aspect ratios were fabricated. 100 ml of aqueous solution containing 2.0×10⁻² M FeCl₃ and 3.0×10⁻⁴ M KH₂PO₄ were aged at 100° C. for 72 hours. The resulting precipitate was centrifuged and washed three times with water. The spindle-shaped hematite particles were dispersed ultrasonically to an 80 ml solution containing PVP (0.6 g), water (6 ml), and ethanol (74 ml). The suspension was stirred using a magnetic stir bar at room temperature and a solution of TEOS (270 μl) in 20 ml ethanol was added, followed by 4 ml of ammonia hydroxide. After 3 h, the reaction mixture was precipitated by centrifuging at 4000 rpm for 16 min. The particles were washed three times with ethanol and centrifuged to collect the product. These silica coated hematite nanoparticles were then suspended in 180 ml distilled water with 1.8 g PVP and 11.34 g oxalic acid (0.5 M) and incubated at 60° C. for 17 h in order to dissolve the hematite core. The silica shell particles were collected by centrifugation and rinsed with DI water twice. The obtained hollow nanoshells were resuspended with 3 ml Gd(NO₃)₃, 0.94 ml Tb(NO₃)₃ (80 mM) or 1.5 ml Eu(NO₃)₃ (80 mM), and 1.8 g PVP in pure water to form 300 ml of solution. 18 g of urea was added to the solution and the solution temperature was maintained at 80° C. for 60 min to form Gd(OH)₃ nanocapsules. These nanocapsules were collected by centrifugation and calcined in a furnace at 600° C. for 60 min to form Gd₂O₃ nanocapsules. The powder was then transferred to a tube furnace with a sulphur/argon flow at 900° C. for 60 min to form Gd₂O₂S nanocapsules. The sulphur reaction significantly increased the nanocapsules' radioluminescence intensity. The obtained nanocapsules were incubated in distilled water (2.5 mg/ml) at 100° C. for 2 h prior to use.

### Preparation of Polyelectrolyte Multilayer Coating

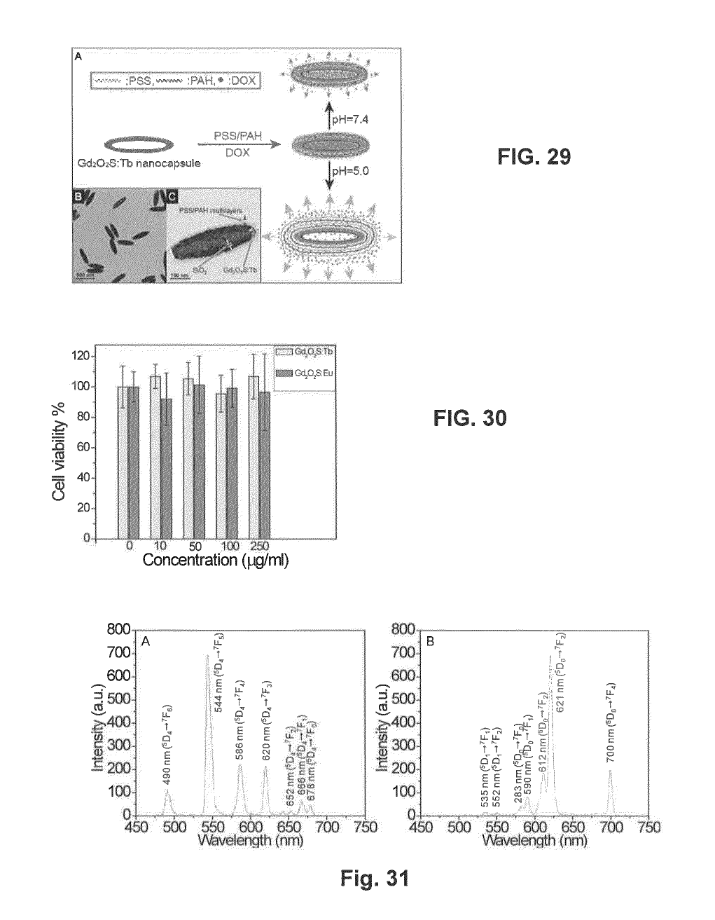

Styrenesulfonate sodium (PSS) and poly(allylamine hydrochloride) (PAH) are widely used polyelectrolytes in pH-controlled release systems. In order to create a stimuli-responsive system for doxorubicin, the X-ray luminescent nanocapsules were coated with eight layers of negative charged PSS and seven layers of positive charged PAH to encapsulate the doxorubicin with layer by layer assembly as schematically illustrated in FIG. 29 (the particle is denoted as DOX@Gd₂O₂S:Tb@PSS/PAH). Because the surface of the nanocapsules are positively charged (+14.9 mV), the first layer of polyelectrolyte coated on the nanocapsules was PSS. After the layer by layer coating, the nanocapsules were coated to an average of 30 nm thick polyelectrolyte with a layer of PSS on the surface.

Specifically, 2 ml of PSS with concentration of 5 mg mL⁻¹ in 0.5 M NaCl was added to a 10 ml aqueous suspension (pH 6) of 100 mg doxorubicin and 200 mg nanocapsules (Gd₂O₂S:Tb). After ultrasonic treatment for 10 min, the suspension was collected by centrifugation and washed three times in distilled water. Gentle shaking followed by ultrasonic treatment for 1 min was used to disperse the particles after centrifugation. Then, 2 ml oppositely charged PAH (5 mg mL-1 in 0.5 M NaCl) was coated on the particles. The PSS coating process was repeated eight times and the PAH coating was repeated 7 times alternately. Finally a composite of doxorubicin-nanocapsules coated with PAH/PSS multilayers was obtained.

### Characterization Methods

Transmission and scanning electron microscopy (TEM) were performed on a H9500 operated at 200 kV and HD2000 microscope operated at 20 kV, respectively. Powder XRD patterns were obtained on a Rigaku diffractometer at 40 kV and 40 mA (CuKα radiation). For fluorescence spectra, 480 nm light was used to excite the scintillators. To measure radioluminescence, X-ray was generated by a mini X-ray tube (Amptek Inc. MA, USA), the X-ray tube was operated with tube voltage of 40 kV and tube current of 40 mA. The sample was mounted on a Leica Microscope (Leica DMI 5000M, Wetzlar, Germany) equipped with a DeltaNu DNS 300 spectrometer (Intevac-DeltaNu, Laramie, Wyo. USA) with a 150 lines/mm grating blazed at 500 nm and with a cooled CCD camera (iDUS-420BV, Andor, South Windsor, Conn.). X-ray luminescence images were captured with an IVIS Lumina-XR Imaging System (Caliper Life Sciences, Hopkinton, Mass., US). Bright field and fluorescent images were taken on a Nikon microscope (Eclipse Ti, Nikon, Melville, N.Y. USA). Determination of the Zeta-potential of the nanoparticles was performed via a Zetasizer Nano ZS (with a 633 nm He—Ne laser) from Malvern Instrument. Prior to the experiment, the particles were diluted in distilled water (0.1 mg/ml). Magnetization measurements were performed at the designated temperature using vibrating sample magnetometer (VSM) option of physical property measurement system (PPMS, Quantum Design, USA), with the applied magnetic field sweeping between +/−3.0 Tesla at a rate of 50 Oe/sec. Determination of the gadolinium content in a sample was performed by inductively coupled plasma (ICP)—(Optima 3100 RL; Perkin-Elmer). All MRI experiments were performed on a Varian 4.7T horizontal bore imaging system (Agilent Inc, Santa Clara, Calif.). Samples, contained in 5 mm NMR tubes, were placed in a 63 mm inner diameter quadrature RF coil for imaging.

### In Vitro HPLC Drug-Release Study and Real-Time Drug Release Tracking

100 μl of nanocapsules with polyelectrolyte mutilayers (10 mg/ml) encapsulating doxorubicin were suspended with release media (7 ml) at pH 5.0 and 7.4 in Slide-A-Lyzer MINI dialysis units at room temperature. The release medium was removed for analysis at given time intervals, and replaced with the same volume of fresh release medium. The doxorubicin concentration was measured with high performance liquid chromatography (HPLC) on a Waters system using an Alltima C18 column (250×4.6 mm, 5 μm).

### Radioluminescence Drug Release Tracking

2 ml of nanocapsules with polyelectrolyte multilayers (25 mg/ml) encapsulating doxorubicin were magnetically stirred at a rate of 400 rpm in release media of either pH 5 or pH 7.4. 50 μl of the solution was taken out for X-ray luminescence analysis without any separation at given time intervals.

### Preparation of Nanocapsules for MR Imaging

T₂ and T₂. MR measurements were acquired for the spindle-shaped SiO₂@Gd₂O₂S:Eu and Gd₂O₂S:Tb particles at a series of concentrations (0.8 mg/ml, 0.4 mg/ml, 0.1 mg/ml, and 0.05 mg/ml). The particles were dispersed in 0.5% agarose gel at 80° C. and cooled to room temperature in NRM tubes to set the gel. The gel prevented settling and aggregation allowing MRI imaging several days after preparation.

### Cell Viability Test

MCF-7 breast cancer cells were seeded at a density of 10,000 cells/well in a 96-well plate. Cells were stored at 37° C. at 5% CO₂ and attached to the plate overnight. Nanocapsules were suspended in media, sonicated for 10 minutes to disperse, and diluted to 250, 100, 50, and 10 μg/ml. Media was removed from wells and fresh media or nanoparticle in media was added to each well. Five repeats were done for each concentration. Nanoparticles were incubated with cells overnight and the next day a Presto Blue assay (Life Technologies) was performed. Media was removed and 100 μl of a 1:9 ratio Presto Blue in culture media was added to each well. Cells were incubated at 37° C. and 5% CO₂ for 45 minutes. Fluorescent intensity was taken with a plate reader with an excitation wavelength of 560 nm and an emission wavelength of 590 nm. Fluorescent intensity for each concentration of nanoparticle was normalized as a percentage of the fluorescent intensity of the control cells. Percent viability averages were plotted with error bars of one standard deviation.

### Results

Particle size distribution showed a narrow distribution of nanocapsules with an average length of 420+/−20 nm and width of 130 nm+/−15 nm. The nanocapsules possessed a 10 nm thick inner silica shell and a 25 nm thick outer Gd₂O₂S:Tb radioluminescent shell with porous morphology. By varying the synthesis condition of the α-Fe₂O₃ template, the length of the templates could be tuned from 14 nm to 600 nm and the aspect ratio could be adjusted from spheres to prolate spheroids. The tunable size range and the morphology make these nanocapsules promising as drug carriers. In addition, the cell viability of the X-ray phosphors on MCF-7 breast cancer cells showed that cell viability is greater than 90% when the concentration of Gd₂O₂S:Tb and Gd₂O₂S:Eu is as high as 250 μg/ml, even after incubation for 24 h (FIG. 30).

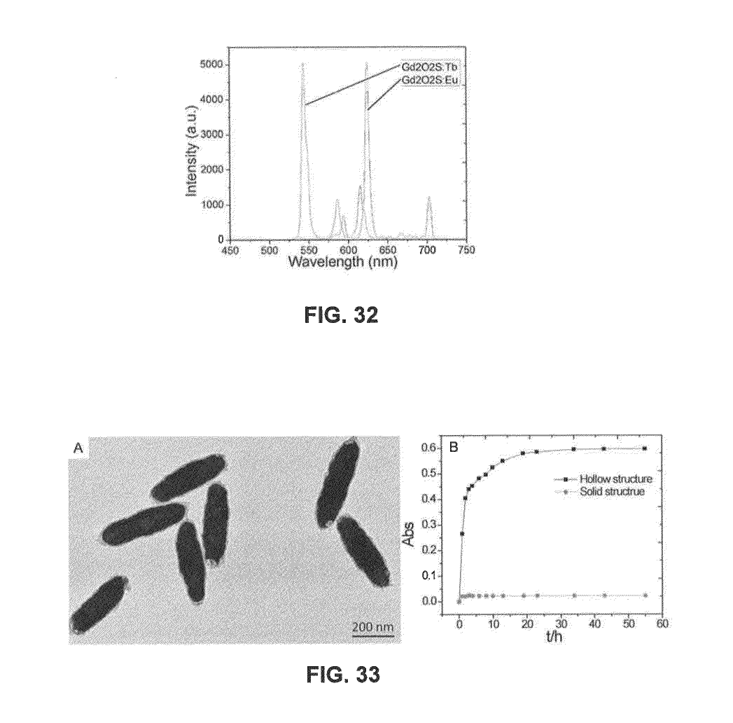

The radioluminescence spectra of nanocapsules (Gd2O2S:Tb, Eu) is presented in FIG. 31 at A and B, respectively. The mechanism of the radioluminescence involves the generation of electron-hole pairs in the host lattice following X-ray absorption. These electron-hole pairs then excite Tb³⁺ and Eu³⁺ centers which emit visible and near infrared light. The conversion efficiency is 60,000-70,000 visible photons/MeV X-ray photon in bulk Gd₂O₂S:Eu, corresponding to an energy efficiency of 15%. The narrow luminescent peaks of Gd₂O₂S:Tb are attributed to the transitions from the ⁵D₄ excited-state to the ⁷F_{J} (J=6, 5, 4, 3, 2, 1, 0) ground states of the Tb³⁺ ion. The ⁵D₄→⁷F₅ transition at 544 nm is the most prominent group. The ⁵D_{0,1}→⁷F_{J} (J=0, 1, 2, 4) transition lines of the Eu³⁺ ions generate the intense peak at 590, 612, 620, 720 nm. The strongest red emission which splits into two peaks at 621 and 612 nm arises from the forced electric-dipole ⁵D₀→⁷F₂ transitions of the Eu³⁺ ions. These nanocapsules displayed similar fluorescence spectra under blue excitation light (460-495 nm) (FIG. 32).

In order to demonstrate that the doxorubicin was loaded into the nanocapsules, nanocapsules with a solid core were synthesized by using the silica coated hematite instead of hollow silica shells as the template. The same doxorubicin loading and polyelectrolyte coating were employed to the nanocapsules with a solid core (iron sulfide) (FIG. 33 at A). From the released doxorubicin from these solid particles, it was calculated that the hollow particles released approximately 20 times more doxorubicin than the solid-core particles, indicating that most of the doxorubicin was stored in the core of the hollow particles (FIG. 33 at B).

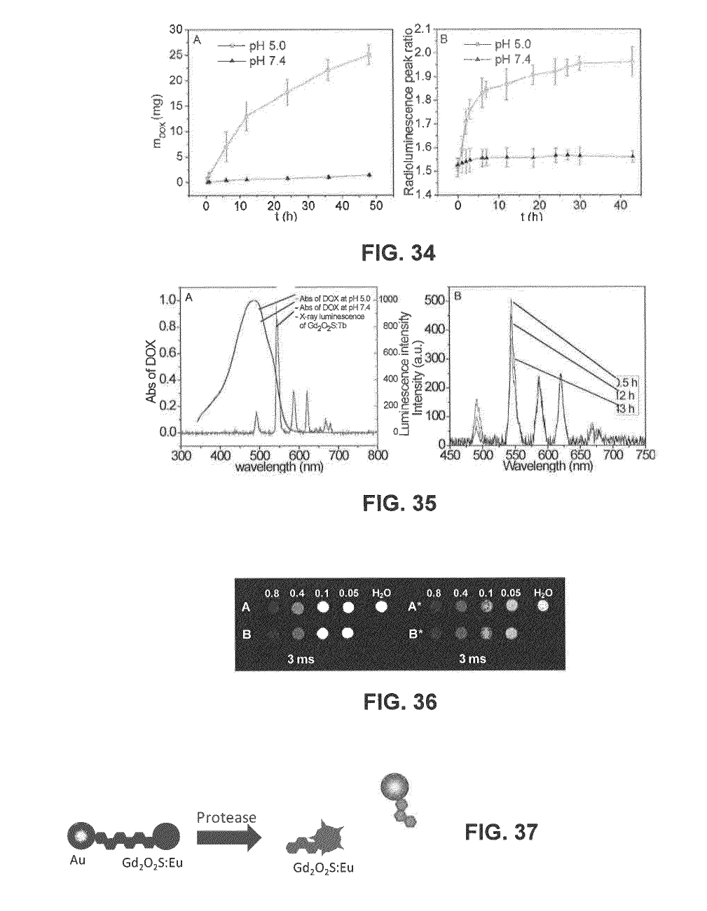

To imitate normal physiological and cancer environments, the pH-response release process was studied at pH 7.4 and 5.0. The cumulative release profile of doxorubicin from the nanocapsules was pH-dependent (FIG. 34 at A). The drug release was enhanced at pH 5.0 which is applicable for cancer therapy due to the low pH environment in tumor endosomes.

The pH-responsive controlled release system was also able to monitor the release process of doxorubicin at different pH by detecting the radioluminescence of Gd₂O₂S:Tb nanocapsules (FIG. 34 at B). At pH 5.0 and 7.4, doxorubicin has the same and broad absorption of light from 350 to 600 nm which overlap some of the X-ray excited luminescent peaks of Gd₂O₂S:Tb (FIG. 35 at A). FIG. 35 at B shows the intensity ratio of X-ray luminescence at 544 nm and 620 nm increases with the release of doxorubicin because doxorubicin absorbs more light at 544 nm than that of 620 nm. The peak intensity ratio reached a maximum value when the doxorubicin concentration in the particles was in equilibrium with the solution concentration.

X-ray luminescent imaging of the Gd₂O₂S:Eu nanocapsules in MCF-7 cancer cells was performed to demonstrate the drug delivery tracking at the cell level. The internalized nanocapsules were brightly luminescent under X-ray radiation. The fluorescence signal of the Gd₂O₂S:Eu nanocapsules in MCF-7 cancer cells after multiple washing steps to eliminate nanocapsules from the cell culture media was clearly seen (image not shown).

In order to demonstrate the application of drug tracking and monitoring in vivo, the Gd₂O₂S:Eu nanocapsules with red and near-infrared radioluminescence were chosen and injected into a mouse after coating with PSS/PAH multilayers. The nanocapsule accumulation was obvious under X-ray. Compared to the Gd₂O₂S:Eu without PSS/PAH mutilayers, the accumulation rate for polyelectrolyte coated nanocapsules was slow during the first hour. Images of organs under X-ray (not shown) confirmed the nanocapsules accumulated in the liver and spleen.

The luminescent nanocapsules mainly consist of gadolinium oxysulfide (Gd₂O₂S) and have similar magnetic properties with gadolinium oxide, which make them a potential MRI contrast agent and magnetic separation tool. As the strength of the applied magnetic field increases, the linear correlation between the magnetization and the applied magnetic field indicates that both Gd₂O₂S:Tb and Gd₂O₂S:Eu nanocapsules are paramagnetic, with minimal hysteresis and a magnetic susceptibility of 1.2×10⁻⁴ emu g⁻¹ Oe⁻¹ and show no sign of saturation up to applied fields of 30 kOe.

In vitro MR assays were performed (T₂ and T₂* weighted imaging) in 0.5% agarose gel for both types of nanocapsules with a series of concentration (0.8 mg/ml, 0.4 mg/ml, 0.1 mg/ml, and 0.05 mg/ml). FIG. 36 shows T₂ and T₂* weighted images after 3 ms. The proton relaxivities r₂ of the nanocapsules were determined from the longitudinal and transverse relaxation rates at various concentrations. The relaxivities, r₂ and r₂* were 50.3 mM⁻¹ s⁻¹ and 116.0 mM⁻¹ s⁻¹ respectively for Gd₂O₂S:Tb nanocapsules; 51.7 mM⁻¹ s⁻¹ and 116.4 mM⁻¹ s⁻¹ for Gd₂O₂S:Eu nanocapsules.

For these nanocapsules, r₂* is larger than r₂ because r₂ includes contributions from local static field inhomogeneities caused by the magnetic moment of the particles. The difference between r₂ and r₂* may provide more specificity towards the contrast agents. These nanocapsules with a ˜25 nm Gd₂O₂S based shell can work well as T₂ contrast agents.

### Example 6

The synthesis protocol for multimodal radioluminescent/upconversion particles is shown schematically in FIG. 37, and detailed below.

### Synthesis of Gd₂O₂S:Tb Precursor (Gd(OH)CO₃:Tb)

3 ml Gd(NO₃)₃ (1 M) and 125 μl Tb(NO3)3 (200 mM) was added in distilled water to form 2100 ml of solution. 18 g of urea was added to the solution after the solution was heated to 80° C. and the solution was maintained at 80° C. for 80 min. The powder collected by centrifugation and washed three times with distilled water

### Silica Coating

To obtain monodisperse upconversion particles-silica core-shell nanoparticles, the up-conversion particle precursor synthesized above was dispersed ultrasonically to a 142 mL solution of Polyvinylpyrrolidone (PVP) (1.2 g), water (12 mL), and ethanol (130 mIL. The suspension was stirred magnetically at room temperature and a solution of TEOS (0.6 ml) in 50 ml ethanol was added, followed by 8 ml of ammonia hydroxide. After 3 h, the reaction mixture was precipitated by centrifuging, and then washed three times with distilled water to collect the product.

### Partial Dissolution of Silica Coated Precursor

The obtained silica coated up-conversion particles were suspended in 600 mL distilled water with 330 μL CH₃COOH. The solution was stirred and maintained under room temperature for three hours.

### Upconversion Phosphor Precursor (Gd(OH)CO3:Yb, Er) Coating and Sulfidation

The collected particles from previous step was suspended in 300 ml with 1.8 g PVP, 1.5 mL Gd(NO₃)₃ (1 M), 1.405 ml Yb(NO₃)₃ (7.5%, 80 mM), and 0.56 ml Ho(NO₃)₃ (1.5%, 40 mM). 9 g urea was added to the solution after the solution was heated to 80° C. and the solution was maintained at 80° C. for 60 min. The powder collected by centrifugation. The precursors of Gd2O2S:Yb, Ho@silica@Gd₂O₂S:Tb were dried at 80° C. for 12 h and then calcined in the air at 600° C. for 60 min. The obtained powder then was transferred to a tube furnace with a sulfur/Ar gas flow at 900° C. for another hour.

Note that the synthesis method is highly flexible, allowing for a wide variety of different host materials and dopants. For example the host material can be synthesized from Gd³⁺, La³⁺, and Y³⁺, ions, and the doped ion for X-ray luminescence can be Tb, Dy, Sm, Eu, Tm or others. The doped ion for upconversion luminescence can include Yb/Ho, Yb/Er, and Yb/Tm. The core and shell can be X-ray phosphor and upconversion phosphor or vice versa, respectively. The precursor etching step by CH₃COOH can be skipped depending the applications of up and down phosphors. The etching step can be performed using CH₃COOH or other acids such as HCl, HNO₃, H₂SO₄ and so on.

### Results

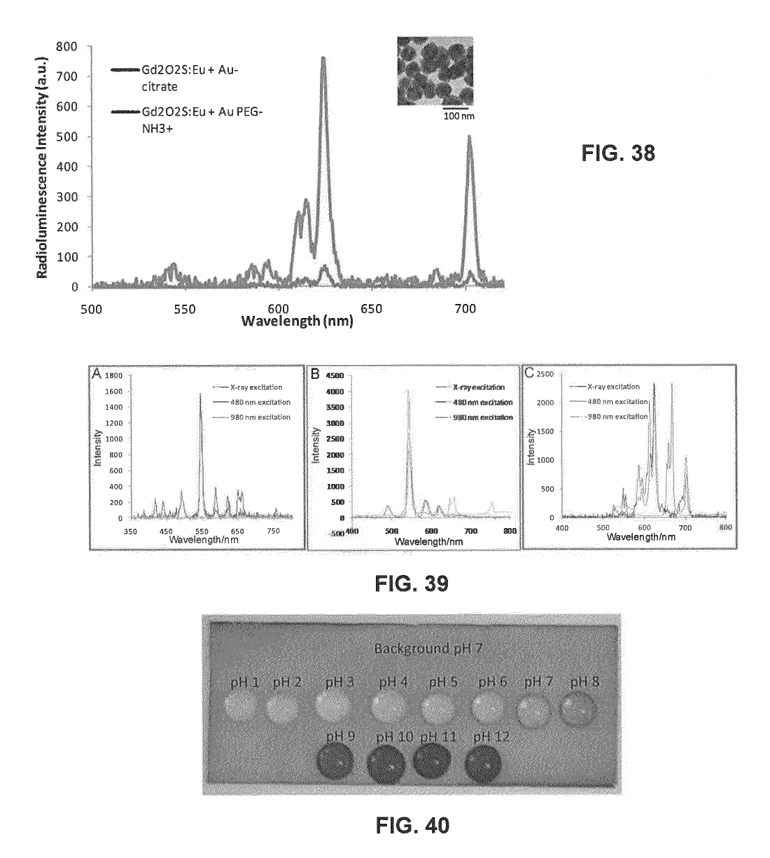

The nanoparticles synthesized as above are spherical particles approximately 50 nm in diameter with a radioluminescent core particle, a hollow space around the core, and a shell particle containing Yb³⁺ ions for upconversion, see TEM image, FIG. 38, inset. The particles luminesce under X-ray excitation, illumination with a 980 nm laser for upconversion, and excitation with blue light. FIG. 39 shows the spectrum under different illumination sources. Such multifunctional particles are useful because they provide multiple methods to image through tissue. The X-ray luminescence allows for high resolution imaging, but requires ionizing radiation. The upconversion functionality provides a complimentary imaging method for lower resolution imaging or long time studies.

### Example 7

### Fabrication of a pH Sensing Film

A hybrid silica sol was prepared by combing tetramethoxysilane (TMOS) with methyltrimethoxysilane (MTMOS) in a mole ratio of 2:1. In a typical preparation, 320 μL of TMOS was mixed with 138 μL MTMOS followed by addition of 230 μL ethanol. A certain amount of water was added to achieve a silane water mole ratio of 1 to 4. Then 166 μL of 0.1 M HCl was added, followed with addition of 40 mg bromocresol green (a pH indicator). The sol was stirred at room temperature for 24 h. Then, 35 μL of the sol was spread onto a precleaned microscope coverslip for 30 s and spin coated for 20 s at 2500 r.p.m to form a uniform and transparent film. In order to achieve a thicker pH sensor film with large absorbance, the pH sensor films are coated again with the same sol-gel. The pH sensor films were dried at room temperature for at least three days before pH sensing. The same process was used to deposit onto microscope slides as shown in FIG. 40.

300 μL of 4 mg/mL upconverting nanoparticles (PVP-YO₂S:Er) in 0.5% carboxymethyl cellulose (CMC) was drop coated to the other side of the pH sensor film. Alternatively, radioluminescent nanoparticles may also be used, or the two particles may be mixed together in the same film for multi-modal exaction. The films with upconversion nanoparticles were dried at room temperature overnight. In order to keep the upconverting nanoparticles in place, a thin layer of PDMS was spread on top. The curing of the polydimethylsiloxane (PDMS) was also at room temperature. The ˜100 μm thick coverslip is transparent which allows optical communication between the luminescent source and the pH indicator. Note, it is recognized that the luminescence from the scintillator layer is emitted in all directions, unless structures are used to guide the light. Thus if the luminescence source is irradiated by a narrow X-ray beam to form a point light source, the light from this this point source spreads out as it passes through the glass and the illuminated a region of the chemical indicating element gets larger as the separation between the luminescence source and indicating layer increase. Since the illuminated region of the chemical indicating element dictates the resolution, the resolution can be improved by bringing the chemical indicating element into closer proximity with the luminescent layer, for example, by coating the chemical indicating layer directly upon a thin luminescent layer, by mixing the luminescent particles with the chemical indicators in the same film, or by fabricating core-shell micro or nanoparticles comprising both indicators and luminescent shells and dispersing these particles into the film. For some applications, a resolution of several hundred micrometers is acceptable, a 100 μm separation between luminescence source and chemical indicating element is not problematic.

### Results

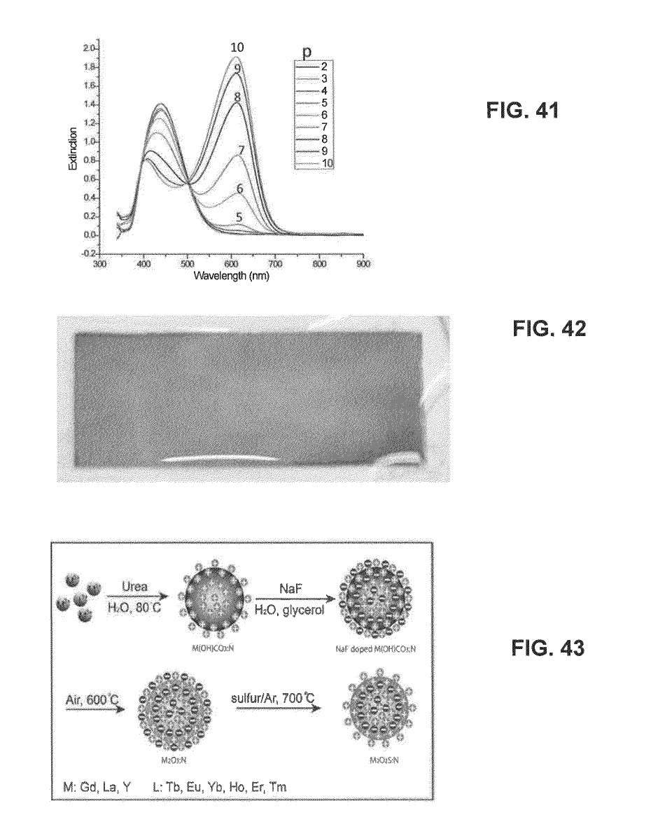

The absorption spectrum of the pH film responds to pH within less than approximately 30 minutes, and has two peaks, an acid peak which absorbs at approximately 450 nm, an a base peak which absorbs at approximately 600 nm, see FIG. 41. This absorption modulates the luminescence spectrum from the upconversion particles on the other side of the coverslip after illumination with a 980 nm laser source. The spectral ratio between 651 nm and 657 nm luminescence was used to form a pH calibration curve. We also observed a pH change as a color change upon culturing bacteria (Staphylococcus epidermidis) on the film as shown in the photo in FIG. 42.

### Example 8

It is recognized that more efficient radioluminescence particles produce stronger signals and are easier to detect through deeper tissue. We have discovered that doping the particles with fluoride ions (as shown schematically in FIG. 43) often increases their radioluminescence (and upconversion) intensities.

Synthesis of Gd2O2S:Eu precursor (Gd(OH)CO3:Eu)

0.5 ml Gd(NO₃)₃ (1 M) and 125 μl Eu(NO₃)₃ (200 mM) was added in distilled water to form 350 ml of solution. 5.25 g of urea was added to the solution after the solution was heated to 80° C. and the solution was maintained at 80° C. for 80 min. The powder collected by centrifugation and washed three times with distilled water.

### NaF Doping

To obtain homogeneous NaF doping in the X-ray scintillators, different amount of NaF was mixed with the above Gd(OH)CO3:Eu in 5 ml distilled. After the solution was stirred for 5 min, 10 ml of glycerol was added to the solution. The solution was maintain at 120° C. for 1.5 h, then at 150° C. for 1 h. The final NaF doped precursor was obtained by centrifuge and washed three time with distilled water.

### Upconversion Phosphor Precursor (Gd(OH)CO3:Yb, Er Coating and Sulfidation

The collected particles from the previous step were calcined in the air at 600° C. for 60 min. The obtained powder then was transferred to a tube furnace with a sulfur/Ar gas flow at 900° C. for another hour.

The host material can be Gd³⁺, La³⁺, and Y³⁺, the doped ion for X-ray luminescence can be Tb, Dy, Sm, Eu and Tm. The doped ion for upconverson luminescence can be Yb/Ho, Yb/Er, and Yb/Tm. The NaF can be replaced by KF, NaBr, KBr.

### Results

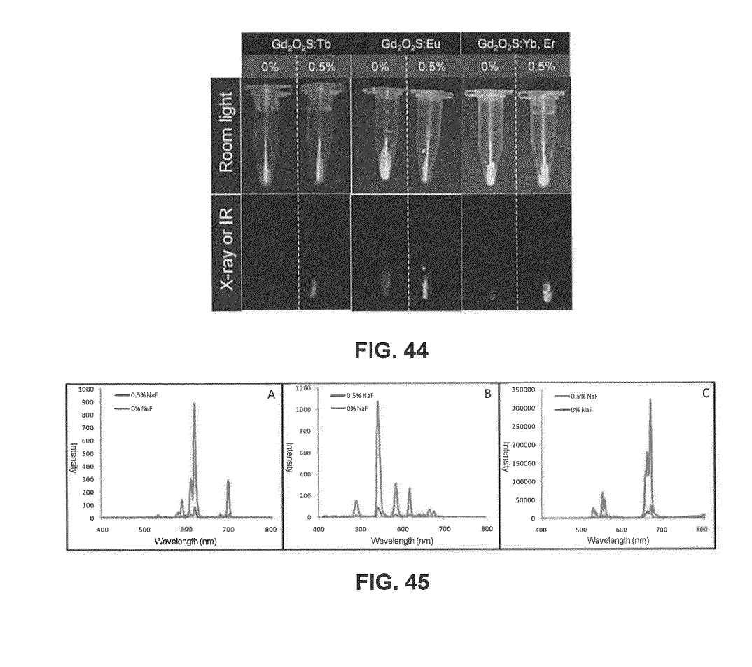

As shown in FIG. 44 and FIG. 45, the luminescence intensity for the Gd₂O₂S:Tb and Gd₂O₂S:Eu nanoparticles increased greatly under X-ray irradiation, while the luminescence intensity for the Gd₂O₂S:Yb,Er increased under 980 nm excitation.

While the subject matter has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present disclosure should be assessed as that of any appended claims and any equivalents thereto.

What is claimed is:

1. A sensing system for imaging of a chemical analyte comprising:
a scintillator that emits optical photons upon excitation by an X-ray; and
a chemical indicating chemical indicating element that is in optical communication with said scintillator, said chemical indicating element modifying the optical spectrum of said scintillator in a manner that depends upon the local concentration of said analyte.

2. The sensing system of claim 1, wherein the scintillator comprises scintillator matrix with a rare earth luminescent dopant.

3. The sensing system of claim 2, wherein the rare earth dopant is europium, terbium, or cerium.

4. The sensing system of claim 1, wherein the scintillator comprises a lanthanide halide.

5. The sensing system of claim 1, wherein the sensing system comprises a scintillator particle with a diameter of from about 5 nm to about 10 μm.

6. The sensing system of claim 5, wherein the particle is a core/shell particle wherein the core comprises the scintillator and the shell comprises the chemical indicating element.

7. The sensing system of claim 5, wherein the particle is a core/shell particle wherein the core comprises the chemical indicating element and the shell comprises the scintillator.

8. The sensing system of claim 7, wherein the core comprises a magnetic material.

9. The sensing system of claim 7, wherein the chemical indicating element containing core encapsulates a drug with an absorption spectrum that overlaps with the scintillator x-ray luminescence spectrum, and said analyte is the drug.

10. The sensing system of claim 9 wherein the shell further comprises a polymer layer with pH-responsive permeability.

11. The sensing system of claim 9 further comprising an organically functionalized layer.

12. The sensing system of claim 11 wherein said organically functionalized layer comprises a polylactic co-glycolic acid) biodegradable layer.

13. The system of claim 9 wherein the surface of said particle is functionalized with a molecular recognition element.

14. The sensing system of claim 9 wherein said particles are embedded within tissue.

15. The sensing system of claim 14 wherein the tissue is at least 1 mm thick.

16. The sensing system of claim 1 further comprising an implanted medical device and polymer film, wherein said scintillator and said chemical indicating element are encapsulated in said polymer film which is coated on the surface of said implanted medical device.

17. The sensing system of claim 16 wherein said polymer film is deposited as two layers, with the bottom layer encapsulating said scintillator and the top layer encapsulating said chemical indicator element.

18. The sensing system of claim 16, wherein said scintillator is a micro- or nano particle and said chemical indicator element is also a micro or nanoparticle, and both said scintillator particle and said chemical indicator particle are encapsulated in said polymer film.

19. The sensing system of claim 16 wherein the film is selected from the group comprising poly dimethyl sulfoxane and polyethylene.

20. The sensing system of claim 16 further comprising a sealed autoclavable bag, said sealed autoclavable bag containing said sensing system.

21. The sensing system of claim 16 further comprising tissue, with said sensing system implanted within said tissue.

22. The sensing system of claim 16 wherein said implantable medical device comprises a fracture fixation plate.

23. The sensing system of claim 16 wherein said chemical indicating element comprises a pH indicator dye.
